(19) United States
(12) Patent Application Publication
(10) Pub. No.: US 2015/0045266 A1
(43) Pub. Date: Feb. 12, 2015

(54) IONIC LIQUID AS LUBRICATING OIL BASE STOCKS, COBASE STOCKS AND MULTIFUNCTIONAL FUNCTIONAL FLUIDS

(71) Applicant: ExxonMobil Research and Engineering Company, Annandale, NJ (US)

(72) Inventors: Abhimanyu Onkar Patil, Westfield, NJ (US); Satish Bodige, Wayne, NJ (US); Tezcan Guney, Ames, IA (US)

(73) Assignee: ExxonMobil Research and Engineering Company, Annandale, NJ (US)

(21) Appl. No.: 14/080,078

(22) Filed: Nov. 14, 2013

Related U.S. Application Data
(60) Provisional application No. 61/737,166, filed on Dec. 14, 2012.

(51) Int. Cl. C10M 105/72; C07C 211/63; C07C 311/48; C07D 233/58

(52) U.S. Cl. CPC C10M 105/72; C07D 233/58; C07C 211/63; C07C 311/48
USPC 508/283; 548/335.1; 548/346.1; 564/82

(57) ABSTRACT

A composition including an ionic liquid alkyl ammonium salt (e.g., tetraalkylammonium cation and bis(trifluoromethanesulfonyl)imide anion) or an ionic liquid imidazolium salt (e.g., 1,3-dialkylimidazolium cation and bis(trifluoromethanesulfonyl)imide anion), that have a structure sufficient to exhibit at least partial solubility in one or more Group I-V base stocks. The disclosure also relates to a lubricating oil base stock and lubricating oil containing the composition, a multifunctional functional fluid containing the composition, and a method for improving solubility of an ionic liquid in a lubricating oil by using as the lubricating oil a formulated oil including a lubricating oil base stock as a major component, and an ionic liquid alkylammonium salt cobase stock, or an ionic liquid imidazolium salt cobase stock, as a minor component.

### CROSS-REFERENCE TO RELATED APPLICATIONS

This application claims priority to U.S. Provisional Application Ser. No. 61/737,166 filed Dec. 14, 2012, herein incorporated by reference in its entirety.

### FIELD

This disclosure relates to compositions that include an ionic liquid alkyl ammonium salt (e.g., tetraalkylammonium cation and bis(trifluoromethanesulfonyl)imide anion) or an ionic liquid imidazolium salt (e.g., 1,3-dialkylimidazolium cation and bis(trifluoromethanesulfonyl)imide anion), a lubricating oil base stock and lubricating oil containing the composition, a multifunctional functional fluid containing the composition, and a method for improving solubility of an ionic liquid in a lubricating oil by using as the lubricating oil a formulated oil comprising a lubricating oil base stock as a major component, and an ionic liquid alkylammonium salt cobase stock, or an ionic liquid imidazolium salt cobase stock, as a minor component.

### BACKGROUND

Ionic liquids are useful as solvents in chemical synthesis, electrochemistry, and other applications due to their ultra-low vapor pressure, non-flammability, and high thermal stability. Ionic liquids are comprised of ions. Conventional ionic liquids include those where the cation is 1-alkyl-3-methylimidazolium, N-alkylpyridinium, or tetraalkylphosphonium. The organic cations, which are generally relatively large compared with simple inorganic cations, account for the low melting points of the salts. Anions range from simple inorganic anions to large complex anions. The synthesis process does not involve high pressures (usually ambient air) or high temperatures (usually 60-80° C.).

Ionic liquids have features that make them attractive for tribological applications, including negligible volatility, non-flammability, high thermal stability, and better intrinsic performance. These characteristics may avoid the need to add expensive additives to facilitate lubrication, as in the case of conventional mineral-oil-based lubricants. Detergents may not be necessary because ionic liquids act as solvents, defoamers may not be necessary due to the ultra-low vapor pressure of ionic liquids, anti-oxidants may not be necessary due to the high thermal stability of ionic liquids, and anti-wear additives may not be necessary if ionic liquids form boundary lubricating films.

Limited publications have shown the potential for using ionic liquids as a new class of lubricants. U.S. Pat. No. 7,754,664 discloses a lubricant or lubricant additive that is an ionic liquid alkylammonium salt. The alkylammonium salt composition comprises an ionic liquid alkylammonium salt represented by the formula RₓNH₍₄₋ₓ₎⁺,[F₃C(CF₂)_{y}S(O)₂⁻]₂N⁻ where x is 1 to 3, wherein R is independently C₁ to C₁₂ straight chain alkyl, branched chain alkyl, cycloalkyl, alkyl substituted cycloalkyl, cycloalkyl substituted alkyl, or, optionally, when x is greater than 1, two R groups comprise a cyclic structure including the nitrogen atom and 4 to 12 carbon atoms, and y is independently 0 to 11.

Ammonium salts of partial esters of phosphoric and thiophosphoric acids are commercially available as extreme pressure and antiwear additives for lubricants and are disclosed in U.S. Pat. Nos. 5,464,549 and 5,942,470. Other patents disclosing ammonium salts of other large anions for lubricants include U.S. Pat. Nos. 3,951,973, 4,115,286 and 4,950,414 where the anions are trithiocyanurate, bis[(mercaptohydrocarbyl)ethylenedioxy]borates, and cyclophosphetane derivatives, respectively.

U.S. Patent Application Publication No. 2009/0270286 discloses a synthetic lubrication oil that comprises an ionic liquid containing an organic cation selected from the group consisting of an imidazolium cation, a pyridinium cation, a quaternary ammonium cation and a quaternary phosphonium cation and a bis(fluorosulfonyl)imide anion, and one comprising an ionic liquid composition which comprises an ionic liquid (A) containing a 1-ethyl-3-methylimidazolium cation and an ionic liquid (B1) containing a 1-methyl-3-propylimidazolium cation and/or an ionic liquid (B2) containing a 1-methyl-3-isopropylimidazolium cation.

However, there remains a need to develop ionic liquids that exhibit superior lubricating properties as the primary lubricant or as lubricant additives, and that also exhibit solubility in conventional base stocks, e.g., Group I-V base stocks. Ionic lubricants with anions that permit superior thermal stability are also desirable for lubricants and lubricant additives.

In particular, there is a need for base stock which would be suitable, for example, for special bearing applications such as for operation at greater than 250° C., where conventional hydrocarbon lubricants start decomposing, but many ionic liquids are stable. Most ionic liquids, however, have little to no solubility (<1%) in nonpolar hydrocarbon oils, e.g., Group I-IV base stocks. Therefore, there is a present need to develop ionic liquids with good solubility in nonpolar lubricating base oils.

The present disclosure provides many additional advantages, which shall become apparent as described below.

### SUMMARY

This disclosure relates in part to a composition comprising:

(i) an ionic liquid alkylammonium salt represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein the ionic liquid alkylammonium salt has a structure sufficient to exhibit at least partial solubility in one or more Group I-V base stocks; or

(ii) an ionic liquid imidazolium salt represented by the formula

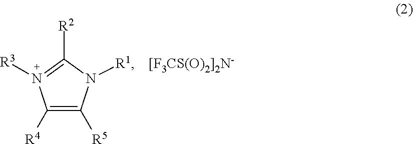

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein the ionic liquid imidazolium salt has a structure sufficient to exhibit at least partial solubility in one or more Group I-V base stocks.

This disclosure also relates in part to a lubricating oil base stock comprising:

(i) an ionic liquid alkylammonium salt represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein the ionic liquid alkylammonium salt has a structure sufficient to exhibit at least partial solubility in one or more Group I-V base stocks; or

(ii) an ionic liquid imidazolium salt represented by the formula

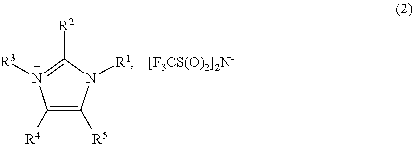

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein the ionic liquid imidazolium salt has a structure sufficient to exhibit at least partial solubility in one or more Group I-V base stocks.

This disclosure further relates in part to a lubricating oil comprising a lubricating oil base stock as a major component, and an ionic liquid alkylammonium salt cobase stock or an ionic liquid imidazolium salt cobase stock, as a minor component; wherein the ionic liquid alkylammonium salt is represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein the ionic liquid alkylammonium salt has a structure sufficient to exhibit at least partial solubility in one or more Group I-V base stocks; and the ionic liquid imidazolium salt is represented by the formula

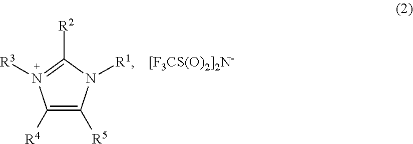

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein the ionic liquid imidazolium salt has a structure sufficient to exhibit at least partial solubility in one or more Group I-V base stocks.

This disclosure yet further relates in part to a multifunctional functional fluid comprising:

(i) an ionic liquid alkylammonium salt represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein the ionic liquid alkylammonium salt has a structure sufficient to exhibit at least partial solubility in one or more Group I-V base stocks; or

(ii) an ionic liquid imidazolium salt represented by the formula

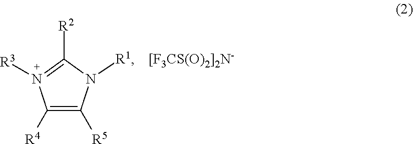

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein the ionic liquid imidazolium salt has a structure sufficient to exhibit at least partial solubility one or more Group I-V base stocks.

This disclosure also relates in part to a method for improving solubility of an ionic liquid in a lubricating oil by using as the lubricating oil a formulated oil comprising a lubricating oil base stock as a major component, and an ionic liquid alkylammonium salt cobase stock or an ionic liquid imidazolium salt cobase stock, as a minor component; wherein the ionic liquid alkylammonium salt is represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein the ionic liquid alkylammonium salt has a structure sufficient to exhibit at least partial solubility in one or more Group I-V base stocks; and the ionic liquid imidazolium salt is represented by the formula

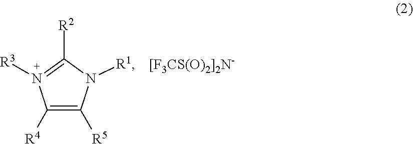

wherein R¹ and R³ are independently a C₁ to C₂₄ straight chain or branched chain alkyl group, a C₆ to C₁₀ aryl group, a C₁ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein the ionic liquid imidazolium salt has a structure sufficient to exhibit at least partial solubility in one or more Group I-V base stocks.

In addition to improved solubility and dispersibility for polar additives and/or sludge generated during service of lubricating oils, improved fuel efficiency can also be attained in an engine lubricated with a lubricating oil by using as the lubricating oil a formulated oil in accordance with this disclosure. The formulated oil comprises a lubricating oil base stock as a major component, and an ionic liquid cobase stock as a minor component. The lubricating oils of this disclosure are particularly advantageous as passenger vehicle engine oil (PVEO) products.

It has been surprisingly found that tetraalkyl ammonium bis(trifluoromethanesulfonyl) imide ionic liquids of this disclosure are soluble in Group V base stocks such as esters and hydrocarbons such as alkylated naphthalene (AN). Most conventional ionic liquids are polar and have little or no solubility (<1%) in nonpolar hydrocarbon oils. The tetraalkyl ammonium bis(trifluoromethanesulfonyl) imide ionic liquids of this disclosure surprisingly exhibit desired base stock properties such as high thermal stability and low volatility in addition to being highly soluble in synthetic base stocks such as esters (Esterex™ A51: di(tridecyl) adipate) and alkylated naphthylene (AN5).

Further, it has been surprisingly found that imidazolium bis(trifluoromethanesulfonyl)imide ionic liquids of this disclosure are highly soluble in Group V base stocks such as esters. Most conventional ionic liquids are polar and have little or no solubility (<1%) in nonpolar hydrocarbon oils. The imidazolium bis(trifluoromethanesulfonyl)imide ionic liquids of this disclosure surprisingly exhibit desired base stock properties such as high thermal stability and low volatility in addition to being highly soluble in synthetic base stocks such as esters (Esterex™ A51: di(tridecyl) adipate).

Further objects, features and advantages of the present disclosure will be understood by reference to the following drawings and detailed description.

### BRIEF DESCRIPTION OF THE DRAWINGS

FIG. 1 sets forth properties of the ionic liquids of Examples 1 and 2 (i.e., Kv at 100° C., Kv at 40° C., viscosity index, solubility in di(tridecyl) adipate ester, solubility in alkylated naphthalene AN5, and thermogravimetric analysis (TGA)).

FIG. 2 sets forth properties of the ionic liquids of Examples 5, 7, 9, 11 and 13 (i.e., Kv at 100° C., Kv at 40° C., viscosity index, solubility in di(tridecyl) adipate ester, solubility in alkylated naphthalene AN5, and thermogravimetric analysis (TGA)).

### DETAILED DESCRIPTION

All numerical values within the detailed description and the claims herein are modified by “about” or “approximately” the indicated value, and take into account experimental error and variations that would be expected by a person having ordinary skill in the art.

### Ionic Liquid Compositions as Base Stocks, Cobase Stocks and Multifunctional Functional Fluids

As indicated above, the compositions of formula (1) of this disclosure comprise an ionic liquid alkylammonium salt represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms. The ionic liquid alkylammonium salt has a structure sufficient to exhibit at least partial solubility in one or more Group I-V base stocks.

Illustrative R substituents include, for example, C₃H₇, C₄H₉, C₅H₁₁, C₆H₁₃, C₈H₁₇, C₁₀H₂₁, C₁₂H₂₅, C₁₄H₂₉, C₁₆H₃₃, and the like. The R substituents can be the same or different.

The compositions of formula (1) of this disclosure have a viscosity (Kv₁₀₀) from 2 to 400 at 100° C., and a viscosity index (VI) from 100 to 300. As used herein, viscosity (Kv₁₀₀) is determined by ASTM D 445-01, and viscosity index (VI) is determined by ASTM D 2270-93 (1998). The compositions of formula (1) of this disclosure have a Noack volatility of no greater than 20 percent, preferably no greater than 18 percent, and more preferably no greater than 15 percent. As used herein, Noack volatility is determined by ASTM D-5800.

Ionic liquids of formula (1) of this disclosure comprise ammonium (e.g., tetraalkylammonium) salts with a bis(perfluoroalkanesulfonyl)imide anion. These ammonium salts display good viscosities for lubricating surfaces at high and low temperatures. These salts display good thermal stability relative to conventional motor oils where the ionic liquid displays an onset of decomposition that is greater than 250° C. These salts display a solubility, preferably at least 5% or greater, more preferably at least 10% or greater, and most preferably at least 15% or greater, in one or more Group I-V base stocks. The melting points of the salts are low, generally below 25° C.

The ammonium salts of formula (1) can be prepared from the appropriate organic amine, R₄N, where R is as defined above for the ammonium salts. The amine is mixed with an equal molar quantity of lithium bis(perfluoroalkanesulfonyl)imide, Li⁺[F₃CS(O)₂]₂N⁻ at room temperature. The addition of a small molar excess of aqueous HCl solution results in the exothermic formation of the desired ammonium salt and lithium chloride as a two layer system. The ammonium salt ionic liquid lower layer is subsequently separated from the top aqueous layer. Multiple washings with deionized water removes LiCl and excess HCl from the ammonium salt ionic liquid. The ammonium salt can be dried by heating under vacuum, for example heating to 70° C. under vacuum for 4 hours.

Illustrative ionic liquid alkylammonium salts of formula (1) of this disclosure can be represented by the formulae

[C₆H₁₃]₄N⁺,[F₃CS(O)₂]₂N⁻

[C₈H₁₇]₄N⁺,[F₃CS(O)₂]₂N⁻

[C₁₀H₂₁]₄N⁺,[F₃CS(O)₂]₂N⁻ and

[C₁₂H₂₅]₄N⁺,[F₃CS(O)₂]₂N⁻.

Preferred ionic liquid alkylammonium salts of formula (1) of this disclosure include tetraoctylammonium bis(trifluoromethanesulfonyl)imide having the formula

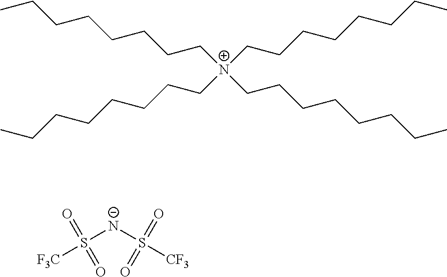 [SMILES: CCCCCCCC[N+](CCCCCCCC)(CCCCCCCC)CCCCCCCC ; O=S(=O)([N-]S(=O)(=O)C(F)(F)F)C(F)(F)F]

tetradecylammonium bis(trifluoromethanesulfonyl)imide having the formula

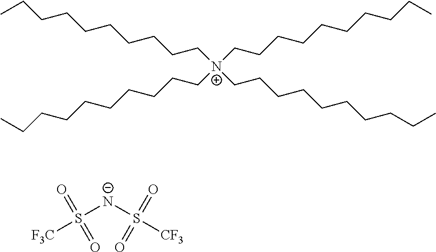 [SMILES: CCCCCCCCCC[N+](CCCCCCCCCC)(CCCCCCCCCC)CCCCCCCCCC ; O=S(=O)([N-]S(=O)(=O)C(F)(F)F)C(F)(F)F]

and the like.

As also indicated above, the compositions of formula (2) of this disclosure comprise an ionic liquid imidazolium salt represented by the formula

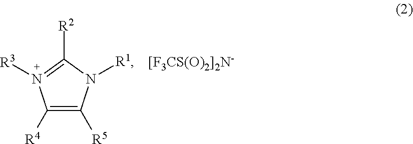

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; and R², R⁴ and R⁵ are hydrogen. The ionic liquid imidazolium salt has a structure sufficient to exhibit at least partial solubility in one or more Group I-V base stocks.

The substituents R¹ to R⁵ may each independently be a hydrogen atom, a halogen atom, a straight chained or branched alkyl group, an alkenyl group, an alkinyl group, an alkoxyl group or an acyl group, which has 1 to 16 carbon atoms, or an amide group, a cyano group, a nitro group, or an amino group, and the alkyl group, the alkenyl group, the alkinyl group, the alkoxyl group and the acyl group may contain a hetero atom selected from N, S and O, and further may contain a conjugate or independent double bond or triple bond.

In a case where the substituents R¹ to R⁵ are an alkyl group, an alkenyl group, an alkinyl group, an alkoxyl group or an acyl group, a carbon atom number thereof is preferably 1 to 16, particularly preferably 1 to 12, and still particularly preferably 1 to 10. Those substituents may be straight chained or branched, and a carbon atom number over the above maximum value is not preferable because of trend of viscosity increase by intermolecular interaction on side chains.

The above alkyl group, alkenyl group, alkinyl group, alkoxyl group and acyl group may contain a hetero atom selected from N, S and O, and the number of the hetero atom to be contained is not specifically limited. Further, they may contain a conjugate or independent double bond or triple bond, and the number of those unsaturated bonds is not specifically limited.

Those alkyl groups are specifically exemplified by a methyl group, an ethyl group, a propyl group, an isopropyl group, a butyl group, an isobutyl group, a secondary butyl group, a tertiary butyl group, a pentyl group, a hexyl group, a cyclopropyl group, a cyclopentyl group, a cyclohexyl group, etc. The alkenyl group is exemplified by a vinyl group, an allyl group, an 1-propenyl group, an isopropenyl group, a 2-butenyl group, an 1,3-butadienyl group, a 2-pentenyl group, a 2-hexenyl group, etc. Further, the alkinyl group is exemplified by an ethynyl group, an 1-propinyl group, a 2-propinyl group, etc., and the alkoxyl group is exemplified by a methoxy group, an ethoxy group, an n-propoxy group, an isopropoxy group, a t-butoxy group, etc., the acyl group is exemplified by an acetyl group, a propionyl group, a butylyl group, a benzoyl group, etc., and the amino group is exemplified by an N,N-dimethylamino group, an N,N-diethylamino group, etc. From a viewpoint of industrial use, easy decomposition by enzymes and increased biodegrability are valuable, and thus an alkoxyl group, an acyl group, an amide group, a cyano group, a nitro group, an amino group, etc. can be mentioned.

As the imidazolium cation shown by the above formula (2), 1,3-substituted imidazolium cation, is preferably used from a viewpoint of easy synthesis. The substituent in the derivatives may be same or different, and a substituent which may contain a multiple bond or a branched chain may be useful.

Preferably, R¹ and R³ are independently a C₁ to C₂₄ straight chain or branched chain alkyl group, a C₆ to C₁₀ aryl group, a C₁ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group. At least one of R¹ and R³ is a C₁₀ to C₂₄ straight chain or branched chain alkyl group. R², R⁴ and R⁵ are preferably hydrogen.

The compositions of formula (2) of this disclosure have a viscosity (Kv₁₀₀) from 2 to 400 at 100° C., and a viscosity index (VI) from 100 to 300. As used herein, viscosity (Kv₁₀₀) is determined by ASTM D 445-01, and viscosity index (VI) is determined by ASTM D 2270-93 (1998). The compositions of formula (2) of this disclosure have a Noack volatility of no greater than 20 percent, preferably no greater than 18 percent, and more preferably no greater than 15 percent. As used herein, Noack volatility is determined by ASTM D-5800.

Ionic liquids of formula (2) of this disclosure comprise imidazolium (e.g., 1,3-substituted imidazolium) salts with a bis(perfluoroalkanesulfonyl)imide anion. These imidazolium salts display good viscosities for lubricating surfaces at high and low temperatures. These salts display good thermal stability relative to conventional motor oils where the ionic liquid displays an onset of decomposition that is greater than 250° C. These salts display a solubility, preferably at least 5% or greater, more preferably at least 10% or greater, and most preferably at least 15% or greater, in one or more Group I-V base stocks. The melting points of the salts are low, generally below 25° C.

The imidazolium salts of formula (2) can be prepared by conventional methods such as an ion exchange method or a metathesis reaction can be applied. For instance, the ionic liquid can be obtained by an anion exchange reaction using a halogenated salt of an organic imidazolium cation to be used and an alkaline metal salt of a bis(fluorosulfonyl)imide anion. The halogen in the halogenated salt is exemplified by chlorine or bromine. The alkaline metal in the alkaline metal salt is exemplified by sodium, potassium, etc.

Amounts of the halogenated salt of the organic imidazolium cation and the alkaline metal salt of a bis(fluorosulfonyl)imide anion to be used in the above reaction are not specifically limited, and 0.5 to 2 equivalents, still preferably 0.8 to 1.2 equivalent of the alkaline metal salt of bis(fluorosulfonyl)imide anion relative to the halogenated salt of the organic imidazolium cation is preferable. In a case of over the above range, economical effect tends to be lowered because the amount over the range does not give influence upon a reaction yield, and in a case of less than the range, on the other hand, a large amount of non-reacted starting material remains to bring tendency of lowering a reaction yield.

Illustrative ionic liquid imidazolium salts of formula (2) of this disclosure can be represented by the formulae

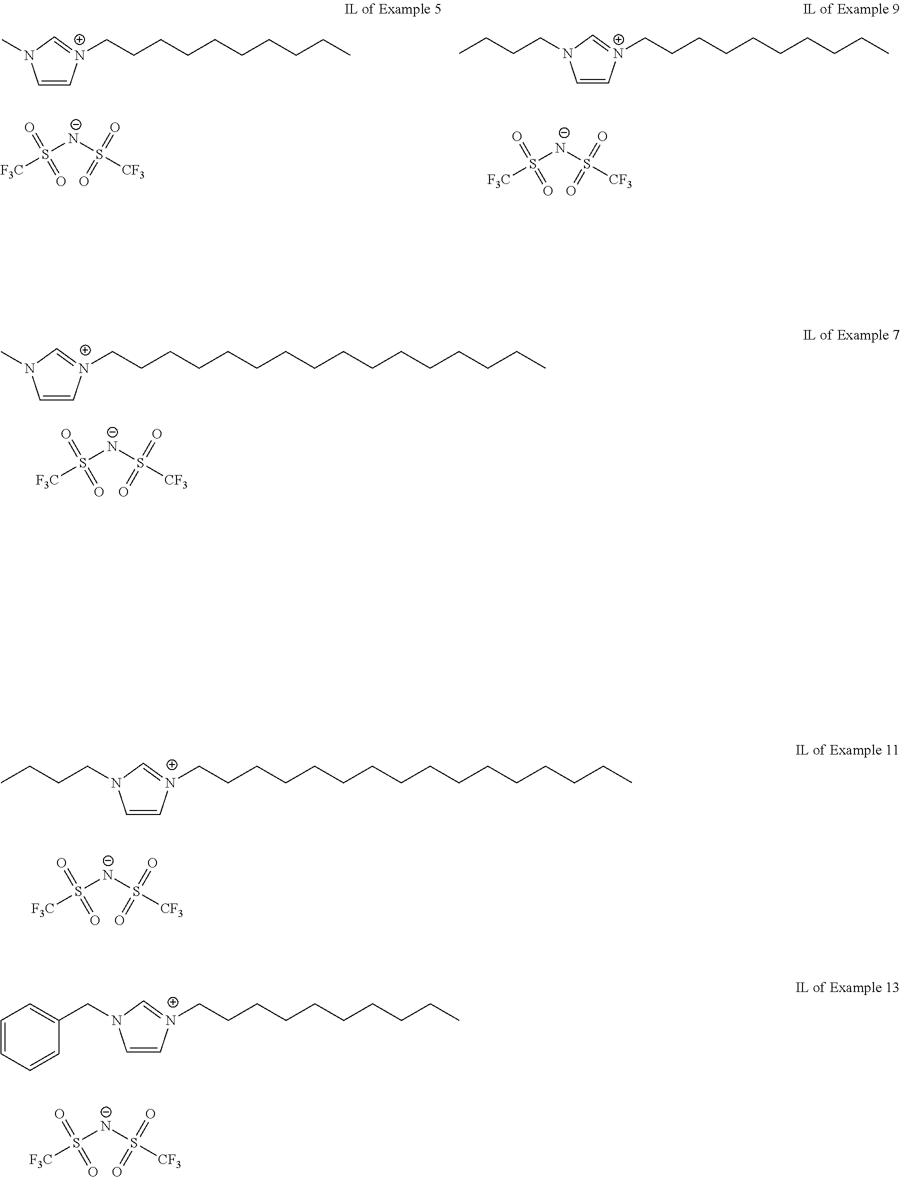

Preferred ionic liquid imidazolium salts of formula (2) of this disclosure include 1-methyl-3-decylimidazolium bis(trifluoromethanesulfonyl)imide having the formula

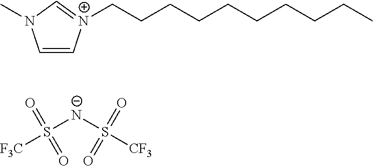 [SMILES: CCCCCCCCCC[n+]1ccn(C)c1 ; O=S(=O)([N-]S(=O)(=O)C(F)(F)F)C(F)(F)F]

1-methyl-3-hexadecylimidazolium bis(trifluoromethanesulfonyl)imide having the formula

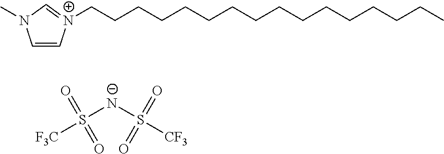 [SMILES: CCCCCCCCCCCCCCCC[n+]1ccn(C)c1 ; O=S(=O)([N-]S(=O)(=O)C(F)(F)F)C(F)(F)F]

1-butyl-3-decylimidazolium bis(trifluoromethanesulfonyl)imide having the formula

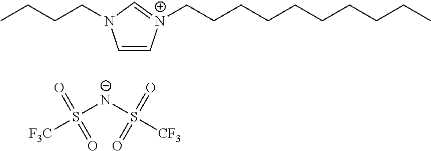 [SMILES: CCCCCCCCCC[n+]1ccn(CCCC)c1 ; O=S(=O)([N-]S(=O)(=O)C(F)(F)F)C(F)(F)F]

1-butyl-3-hexadecylimidazolium bis(trifluoromethanesulfonyl)imide having the formula

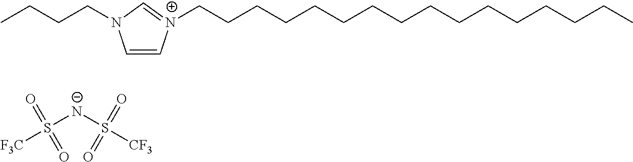 [SMILES: CCCCCCCCCCCCCCCC[n+]1ccn(CCCC)c1 ; O=S(=O)([N-]S(=O)(=O)C(F)(F)F)C(F)(F)F]

1-benzyl-3-decylimidazolium bis(trifluoromethanesulfonyl)imide having the formula

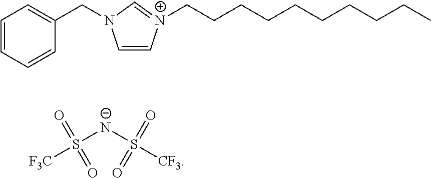 [SMILES: CCCCCCCCCC[n+]1ccn(Cc2ccccc2)c1 ; O=S(=O)([N-]S(=O)(=O)C(F)(F)F)C(F)(F)F]

and the like.

The ionic liquids of this disclosure are organic salts (100% ions) with a melting point below 100° C. exhibiting no measurable vapor pressure below thermal decomposition. The ionic liquids are clear bright synthetic fluids with wide viscosity range (from single digit to >100 cSt) at room temperature. They are liquid over wide temperature range (often over 300° C.), and they don't evaporate like most other liquids. The ionic liquids have low freeing points and their typical structures (imidazolium, pyrrolidium, ammonium, pyridinium, phosphonium, etc.) looks like surface interactive friction/wear type lube additive. Typical properties of ionic liquids include liquid below 100° C., 100% ions (strongly polar), low viscosity, virtually no vapor pressure, thermal and hydrolytic stability, non flammable, regenerative, broad liquid range (>300° C.), ionic liquid properties (viscosity, acidity, basicity, density) can be tunable using cations and anions, and the like.

Advantages of the ionic liquids of this disclosure include: 1) reduced parasitic energy losses by reducing friction, 2) extended service life and maintenance cycle because of wear reduction, 3) expanded high temperature lubricant usage because of high thermal stability and 4) safer transportation and storage because of non-flammability. Thus, the lubricants of this disclosure can improve and replace many lubricants that are currently being used with potential friction and wear reduction.

Ionic liquids are currently in use, for example, in chemical synthesis and separation, food science, cellulose processing, paint formulations. Other potential uses of ionic liquids include, for example, solvents, catalyst/supported catalyst/solvent for catalyst, separation (e.g., gas absorbent/storage/extraction), electrolytes, performance additives (e.g., plasticizers, dispersing agents, compatibilizers, solubilizers, antistatic agents, and the like.

The ionic liquid compositions of this disclosure exhibit unique properties which result from the composite properties of the wide variety of cations and anions. In a comparison of a typical ionic liquid, e.g., 1-ethyl-3-methylimidazolium ethyl sulfate (mp<−20° C.), with a typical inorganic salt, e.g., table salt (NaCl, mp 801° C.), it becomes obvious why there is a difference between them. The ionic liquid has a significantly lower symmetry. Furthermore, the charge of the cation as well as the charge of the anion is distributed over a larger volume of the molecule by resonance. As a consequence, the solidification of the ionic liquid will take place at lower temperatures. In some cases, especially if long aliphatic side chains are involved, a glass transition is observed instead of a melting point.

The strong ionic (Coulomb-) interaction within the ionic liquids of this disclosure results in a negligible vapor pressure (unless decomposition occurs), a non-flammable substance, and in a high thermally, mechanically as well as electrochemically stable product. In addition to this desirable combination of properties, the ionic liquids offer other favorable properties, for example, very appealing solvent properties and immiscibility with water or organic solvents that result in biphasic systems.

The choice of the cation has a strong impact on the properties of the ionic liquid and will often define the stability. The chemistry and functionality of the ionic liquid is, in general, controlled by the choice of the anion. In accordance with this disclosure, the possible combinations of organic cations and anions allows for designing and fine-tuning physical and chemical properties by introducing or combining structural motifs and, thereby, making tailor-made materials and solutions possible.

The ionic liquid is primarily salt or mixture of salts which melts below room temperature. Ionic liquids may be characterized by the general formula Q⁺A⁻, where Q⁺ is quaternary ammonium, quaternary phosphonium, quaternary sulfonium, and A⁻ is a negatively charged ion such as Cl⁻, Br⁻, NO₃⁻, BF₄⁻, BCl₄⁻, PF₆⁻, SbF₆⁻, AlCl₄⁻, CuCl₂⁻, FeCl₃⁻, and the like.

The ionic liquids of this disclosure may provide more significant friction reduction if used as neat basestock or cobasestock. These fluids may establish a tribolayer that is physically adsorbed onto and/or chemically react with the metal surfaces to effectively reduce friction and wear under boundary lubrication.

This disclosure provides lubricating oils useful as engine oils and in other applications characterized by excellent solvency characteristics. The lubricating oils are based on high quality base stocks including a major portion of a hydrocarbon base fluid such as a PAO or GTL with a secondary cobase stock component which is an ionic liquid alkylammonium salt or an ionic liquid imidazolium salt as described herein. The lubricating oil base stock can be any oil boiling in the lube oil boiling range, typically between 100 to 450° C. In the present specification and claims, the terms base oil(s) and base stock(s) are used interchangeably.

In the lubricating oils of this disclosure, the lubricating oil base stock is present in an amount from 50 weight percent to 99 weight percent, preferably from 55 weight percent to 95 weight percent, and more preferably from 60 to 90 weight percent, and the ionic liquid alkylammonium salt cobase stock or the ionic liquid imidazolium salt cobase stock is present in an amount from 1 weight percent to 50 weight percent, preferably from 5 weight percent to 45 weight percent, and more preferably from 10 to 60 weight percent, based on the total weight of the lubricating oil.

The viscosity-temperature relationship of a lubricating oil is one of the critical criteria which must be considered when selecting a lubricant for a particular application. Viscosity Index (VI) is an empirical, unitless number which indicates the rate of change in the viscosity of an oil within a given temperature range. Fluids exhibiting a relatively large change in viscosity with temperature are said to have a low viscosity index. A low VI oil, for example, will thin out at elevated temperatures faster than a high VI oil. Usually, the high VI oil is more desirable because it has higher viscosity at higher temperature, which translates into better or thicker lubrication film and better protection of the contacting machine elements.

In another aspect, as the oil operating temperature decreases, the viscosity of a high VI oil will not increase as much as the viscosity of a low VI oil. This is advantageous because the excessive high viscosity of the low VI oil will decrease the efficiency of the operating machine. Thus high VI (HVI) oil has performance advantages in both high and low temperature operation. VI is determined according to ASTM method D 2270-93 [1998]. VI is related to kinematic viscosities measured at 40° C. and 100° C. using ASTM Method D 445-01.

This disclosure also provides multifunctional functional fluids comprising an ionic liquid alkylammonium salt. The ionic liquid alkylammonium salt is represented by the formula

R₄N⁺,[F₃CS(O)₂]₂N⁻ (1)

wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms. The ionic liquid alkylammonium salt has a structure sufficient to exhibit at least partial solubility in one or more Group I-V base stocks.

This disclosure further provides multifunctional functional fluids comprising an ionic liquid imidazolium salt. The ionic liquid imidazolium salt is represented by the formula

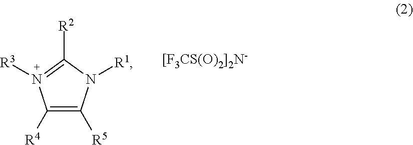

wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.

For ionic liquid base stocks of this disclosure, the ionic liquid alkylammonium salt base stock or the ionic liquid imidazolium salt base stock is present in an amount from 50 weight percent to 99 weight percent, preferably from 55 weight percent to 95 weight percent, and more preferably from 60 to 90 weight percent, of the ionic liquid formulation. For ionic liquid multifunctional functional fluids of this disclosure, the ionic liquid alkylammonium salt base stock or the ionic liquid imidazolium salt base stock is present in an amount from 50 weight percent to 99 weight percent, preferably from 55 weight percent to 95 weight percent, and more preferably from 60 to 90 weight percent, of the fluid.

### Lubricating Oil Base Stocks

A wide range of lubricating oils are known in the art. Lubricating oils that are useful in the present disclosure are both natural oils and synthetic oils. Natural and synthetic oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve the at least one lubricating oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad categories of base oil stocks developed and defined by the American Petroleum Institute (API Publication 1509; www.API.org) to create guidelines for lubricant base oils. Group I base stocks generally have a viscosity index of between 80 to 120 and contain greater than 0.03% sulfur and less than 90% saturates. Group II base stocks generally have a viscosity index of between 80 to 120, and contain less than or equal to 0.03% sulfur and greater than or equal to 90% saturates. Group III stock generally has a viscosity index greater than 120 and contains less than or equal to 0.03% sulfur and greater than 90% saturates. Group IV includes polyalphaolefins (PAO). Group V base stocks include base stocks not included in Groups I-IV. The table below summarizes properties of each of these five groups.

| | Base Oil Properties | | |
|---|---|---|---|
| | Saturates | Sulfur | Viscosity Index |
| Group I | <90 and/or | >0.03% and | ≧80 and <120 |
| Group II | ≧90 and | ≦0.03% and | ≧80 and <120 |
| Group III | ≧90 and | ≦0.03% and | ≧120 |

| | |
|---|---|
| Group IV | Includes polyalphaolefins (PAO) products |
| Group V | All other base oil stocks not included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful in the present disclosure. Natural oils vary also as to the method used for their production and purification, for example, their distillation range and whether they are straight run or cracked, hydrorefined, or solvent extracted.

Group II and/or Group III hydroprocessed or hydrocracked base stocks, as well as synthetic oils such as polyalphaolefins, alkyl aromatics and synthetic esters, i.e. Group IV and Group V oils are also well known base stock oils.

Synthetic oils include hydrocarbon oil such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks, the Group IV API base stocks, are a commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073, which are incorporated herein by reference in their entirety. Group IV oils, that is, the PAO base stocks have viscosity indices preferably greater than 130, more preferably greater than 135, still more preferably greater than 140.

Esters in a minor amount may be useful in the lubricating oils of this disclosure. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols such as the neopentyl polyols; e.g., neopentyl glycol, trimethylol ethane, 2-methyl-2-propyl-1,3-propanediol, trimethylol propane, pentaerythritol and dipentaerythritol with alkanoic acids containing at least 4 carbon atoms, preferably C₅ to C₃₀ acids such as saturated straight chain fatty acids including caprylic acid, capric acids, lauric acid, myristic acid, palmitic acid, stearic acid, arachic acid, and behenic acid, or the corresponding branched chain fatty acids or unsaturated fatty acids such as oleic acid, or mixtures of any of these materials.

Esters should be used in a amount such that the improved wear and corrosion resistance provided by the lubricating oils of this disclosure are not adversely affected.

Non-conventional or unconventional base stocks and/or base oils include one or a mixture of base stock(s) and/or base oil(s) derived from: (1) one or more Gas-to-Liquids (GTL) materials, as well as (2) hydrodewaxed, or hydroisomerized/cat (and/or solvent) dewaxed base stock(s) and/or base oils derived from synthetic wax, natural wax or waxy feeds, mineral and/or non-mineral oil waxy feed stocks such as gas oils, slack waxes (derived from the solvent dewaxing of natural oils, mineral oils or synthetic oils; e.g., Fischer-Tropsch feed stocks), natural waxes, and waxy stocks such as gas oils, waxy fuels hydrocracker bottoms, waxy raffinate, hydrocrackate, thermal crackates, foots oil or other mineral, mineral oil, or even non-petroleum oil derived waxy materials such as waxy materials recovered from coal liquefaction or shale oil, linear or branched hydrocarbyl compounds with carbon number of 20 or greater, preferably 30 or greater and mixtures of such base stocks and/or base oils.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include oils boiling in the lube oil boiling range (1) separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized cat and/or solvent dewaxed synthesized wax or waxy hydrocarbons; (3) hydrodewaxed or hydroisomerized cat and/or solvent dewaxed Fischer-Tropsch (F-T) material (i.e., hydrocarbons, waxy hydrocarbons, waxes and possible analogous oxygenates); preferably hydrodewaxed or hydroisomerized/followed by cat and/or solvent dewaxing dewaxed F-T waxy hydrocarbons, or hydrodewaxed or hydroisomerized/followed by cat (or solvent) dewaxing dewaxed, F-T waxes, or mixtures thereof.

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having kinematic viscosities at 100° C. of from 2 mm²/s to 50 mm²/s (ASTM D445). They are further characterized typically as having pour points of −5° C. to −40° C. or lower (ASTM D97). They are also characterized typically as having viscosity indices of 80 to 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than 10 ppm, and more typically less than 5 ppm of each of these elements. The sulfur and nitrogen content of GTL base stock(s) and/or base oil(s) obtained from F-T material, especially F-T wax, is essentially nil. In addition, the absence of phosphorous and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the formulated lubricating oils useful in the present disclosure are any of the variety of oils corresponding to API Group I, Group II, Group III, Group IV, Group V and Group VI oils and mixtures thereof, preferably API Group II, Group III, Group IV, Group V and Group VI oils and mixtures thereof, more preferably the Group III to Group VI base oils due to their exceptional volatility, stability, viscometric and cleanliness features. Minor quantities of Group I stock, such as the amount used to dilute additives for blending into formulated lube oil products, can be tolerated but should be kept to a minimum, i.e. amounts only associated with their use as diluent/carrier oil for additives used on an “as received” basis. Even in regard to the Group II stocks, it is preferred that the Group II stock be in the higher quality range associated with that stock, i.e. a Group II stock having a viscosity index in the range 100<VI<120.

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) and hydrodewaxed, or hydroisomerized/cat (and/or solvent) dewaxed base stock(s) and/or base oil(s) typically have very low sulfur and nitrogen content, generally containing less than 10 ppm, and more typically less than 5 ppm of each of these elements. The sulfur and nitrogen content of GTL base stock(s) and/or base oil(s) obtained from F-T material, especially F-T wax, is essentially nil. In addition, the absence of phosphorous and aromatics make this material especially suitable for the formulation of low sulfur, sulfated ash, and phosphorus (low SAP) products.

The basestock component of the present lubricating oils will typically be from 50 to 99 weight percent of the total composition (all proportions and percentages set out in this specification are by weight unless the contrary is stated) and more usually in the range of 80 to 99 weight percent.

### Other Additives

The formulated lubricating oil useful in the present disclosure may additionally contain one or more of the other commonly used lubricating oil performance additives including but not limited to dispersants, other detergents, corrosion inhibitors, rust inhibitors, metal deactivators, other anti-wear agents and/or extreme pressure additives, anti-seizure agents, wax modifiers, viscosity index improvers, viscosity modifiers, fluid-loss additives, seal compatibility agents, other friction modifiers, lubricity agents, anti-staining agents, chromophoric agents, defoamants, demulsifiers, emulsifiers, densifiers, wetting agents, gelling agents, tackiness agents, colorants, and others. For a review of many commonly used additives, see Klamann in Lubricants and Related Products, Verlag Chemie, Deerfield Beach, Fla.; ISBN 0-89573-177-0. Reference is also made to “Lubricant Additives Chemistry and Applications” edited by Leslie R. Rudnick, Marcel Dekker, Inc. New York, 2003 ISBN: 0-8247-0857-1.

The types and quantities of performance additives used in combination with the instant disclosure in lubricant compositions are not limited by the examples shown herein as illustrations.

### Viscosity Improvers

Viscosity improvers (also known as Viscosity Index modifiers, and VI improvers) increase the viscosity of the oil composition at elevated temperatures which increases film thickness, while having limited effect on viscosity at low temperatures.

Suitable viscosity improvers include high molecular weight hydrocarbons, polyesters and viscosity index improver dispersants that function as both a viscosity index improver and a dispersant. Typical molecular weights of these polymers are between 10,000 to 1,000,000, more typically 20,000 to 500,000, and even more typically between 50,000 and 200,000.

Examples of suitable viscosity improvers are polymers and copolymers of methacrylate, butadiene, olefins, or alkylated styrenes. Polyisobutylene is a commonly used viscosity index improver. Another suitable viscosity index improver is polymethacrylate (copolymers of various chain length alkyl methacrylates, for example), some formulations of which also serve as pour point depressants. Other suitable viscosity index improvers include copolymers of ethylene and propylene, hydrogenated block copolymers of styrene and isoprene, and polyacrylates (copolymers of various chain length acrylates, for example). Specific examples include styrene-isoprene or styrene-butadiene based polymers of 50,000 to 200,000 molecular weight.

The amount of viscosity modifier may range from zero to 8 wt %, preferably zero to 4 wt %, more preferably zero to 2 wt % based on active ingredient and depending on the specific viscosity modifier used.

### Antioxidants

Typical antioxidant include phenolic antioxidants, aminic antioxidants and oil-soluble copper complexes.

The phenolic antioxidants include sulfurized and non-sulfurized phenolic antioxidants. The terms “phenolic type” or “phenolic antioxidant” used herein includes compounds having one or more than one hydroxyl group bound to an aromatic ring which may itself be mononuclear, e.g., benzyl, or poly-nuclear, e.g., naphthyl and spiro aromatic compounds. Thus “phenol type” includes phenol per se, catechol, resorcinol, hydroquinone, naphthol, etc., as well as alkyl or alkenyl and sulfurized alkyl or alkenyl derivatives thereof, and bisphenol type compounds including such bi-phenol compounds linked by alkylene bridges sulfuric bridges or oxygen bridges. Alkyl phenols include mono- and poly-alkyl or alkenyl phenols, the alkyl or alkenyl group containing from 3-100 carbons, preferably 4 to 50 carbons and sulfurized derivatives thereof, the number of alkyl or alkenyl groups present in the aromatic ring ranging from 1 to up to the available unsatisfied valences of the aromatic ring remaining after counting the number of hydroxyl groups bound to the aromatic ring.

Generally, therefore, the phenolic anti-oxidant may be represented by the general formula:

(R)ₓ—Ar—(OH)_{y}

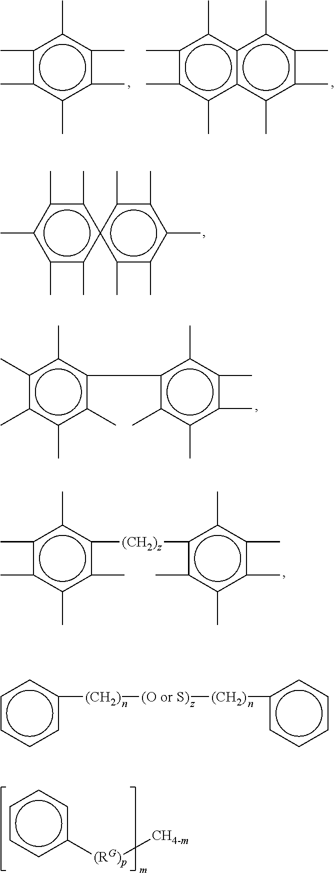

where Ar is selected from the group consisting of:
wherein R is a C₃-C₁₀₀ alkyl or alkenyl group, a sulfur substituted alkyl or alkenyl group, preferably a C₄-C₅₀ alkyl or alkenyl group or sulfur substituted alkyl or alkenyl group, more preferably C₃-C₁₀₀ alkyl or sulfur substituted alkyl group, most preferably a C₄-C₅₀ alkyl group, R^{G} is a C₁-C₁₀₀ alkylene or sulfur substituted alkylene group, preferably a C₂-C₅₀ alkylene or sulfur substituted alkylene group, more preferably a C₂-C₂ alkylene or sulfur substituted alkylene group, y is at least 1 to up to the available valences of Ar, x ranges from 0 to up to the available valances of Ar-y, z ranges from 1 to 10, n ranges from 0 to 20, and m is 0 to 4 and p is 0 or 1, preferably y ranges from 1 to 3, x ranges from 0 to 3, z ranges from 1 to 4 and n ranges from 0 to 5, and p is 0.

Preferred phenolic antioxidant compounds are the hindered phenolics and phenolic esters which contain a sterically hindered hydroxyl group, and these include those derivatives of dihydroxy aryl compounds in which the hydroxyl groups are in the o- or p-position to each other. Typical phenolic anti-oxidants include the hindered phenols substituted with C₁+ alkyl groups and the alkylene coupled derivatives of these hindered phenols. Examples of phenolic materials of this type 2-t-butyl-4-heptyl phenol; 2-t-butyl-4-octyl phenol; 2-t-butyl-4-dodecyl phenol; 2,6-di-t-butyl-4-heptyl phenol; 2,6-di-t-butyl-4-dodecyl phenol; 2-methyl-6-t-butyl-4-heptyl phenol; 2-methyl-6-t-butyl-4-dodecyl phenol; 2,6-di-t-butyl-4 methyl phenol; 2,6-di-t-butyl-4-ethyl phenol; and 2,6-di-t-butyl 4 alkoxy phenol; and

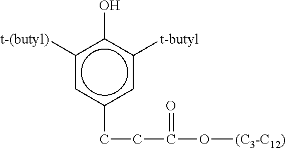

Phenolic type antioxidants are well known in the lubricating industry and commercial examples such as Ethanox® 4710, Irganox® 1076, Irganox® L1035, Irganox® 1010, Irganox® L109, Irganox® L118, Irganox® L135 and the like are familiar to those skilled in the art. The above is presented only by way of exemplification, not limitation on the type of phenolic antioxidants which can be used.

The phenolic antioxidant can be employed in an amount in the range of 0.1 to 3 wt/o, preferably 1 to 3 wt %, more preferably 1.5 to 3 wt % on an active ingredient basis.

Aromatic amine antioxidants include phenyl-α-naphthyl amine which is described by the following molecular structure:

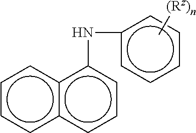

wherein R^{z} is hydrogen or a C₁ to C₁₄ linear or C₃ to C₁₄ branched alkyl group, preferably C₁ to C₁₀ linear or C₃ to C₁₀ branched alkyl group, more preferably linear or branched C₆ to C₈ and n is an integer ranging from 1 to 5 preferably 1. A particular example is Irganox L06.

Other aromatic amine antioxidants include other alkylated and non-alkylated aromatic amines such as aromatic monoamines of the formula R⁸R⁹R¹⁰N where R⁸ is an aliphatic, aromatic or substituted aromatic group, R⁹ is an aromatic or a substituted aromatic group, and R¹⁰ is H, alkyl, aryl or R¹¹S(O)xR¹² where R¹¹ is an alkylene, alkenylene, or aralkylene group, R¹² is a higher alkyl group, or an alkenyl, aryl, or alkaryl group, and x is 0, 1 or 2. The aliphatic group R⁸ may contain from 1 to 20 carbon atoms, and preferably contains from 6 to 12 carbon atoms. The aliphatic group is a saturated aliphatic group. Preferably, both R⁸ and R⁹ are aromatic or substituted aromatic groups, and the aromatic group may be a fused ring aromatic group such as naphthyl. Aromatic groups R⁸ and R⁹ may be joined together with other groups such as S.

Typical aromatic amines antioxidants have alkyl substituent groups of at least 6 carbon atoms. Examples of aliphatic groups include hexyl, heptyl, octyl, nonyl, and decyl. Generally, the aliphatic groups will not contain more than 14 carbon atoms. The general types of such other additional amine antioxidants which may be present include diphenylamines, phenothiazines, imidodibenzyls and diphenyl phenylene diamines. Mixtures of two or more of such other additional aromatic amines may also be present. Polymeric amine antioxidants can also be used.

Another class of antioxidant used in lubricating oil compositions and which may also be present are oil-soluble copper compounds. Any oil-soluble suitable copper compound may be blended into the lubricating oil. Examples of suitable copper antioxidants include copper dihydrocarbyl thio- or dithio-phosphates and copper salts of carboxylic acid (naturally occurring or synthetic). Other suitable copper salts include copper dithiacarbamates, sulphonates, phenates, and acetylacetonates. Basic, neutral, or acidic copper Cu(I) and or Cu(II) salts derived from alkenyl succinic acids or anhydrides are know to be particularly useful.

Such antioxidants may be used individually or as mixtures of one or more types of antioxidants, the total amount employed being an amount of 0.50 to 5 wt %, preferably 0.75 to 3 wt % (on an as-received basis).

### Detergents

In addition to the alkali or alkaline earth metal salicylate detergent which is an essential component in the present disclosure, other detergents may also be present. While such other detergents can be present, it is preferred that the amount employed be such as to not interfere with the synergistic effect attributable to the presence of the salicylate. Therefore, most preferably such other detergents are not employed.

If such additional detergents are present, they can include alkali and alkaline earth metal phenates, sulfonates, carboxylates, phosphonates and mixtures thereof. These supplemental detergents can have total base number (TBN) ranging from neutral to highly overbased, i.e. TBN of 0 to over 500, preferably 2 to 400, more preferably 5 to 300, and they can be present either individually or in combination with each other in an amount in the range of from 0 to 10 wt %, preferably 0.5 to 5 wt % (active ingredient) based on the total weight of the formulated lubricating oil. As previously stated, however, it is preferred that such other detergent not be present in the formulation.

Such additional other detergents include by way of example and not limitation calcium phenates, calcium sulfonates, magnesium phenates, magnesium sulfonates and other related components (including borated detergents).

### Dispersants

During engine operation, oil-insoluble oxidation byproducts are produced. Dispersants help keep these byproducts in solution, thus diminishing their deposition on metal surfaces. Dispersants may be ashless or ash-forming in nature. Preferably, the dispersant is ashless. So-called ashless dispersants are organic materials that form substantially no ash upon combustion. For example, non-metal-containing or borated metal-free dispersants are considered ashless. In contrast, metal-containing detergents discussed above form ash upon combustion.

Suitable dispersants typically contain a polar group attached to a relatively high molecular weight hydrocarbon chain. The polar group typically contains at least one element of nitrogen, oxygen, or phosphorus. Typical hydrocarbon chains contain 50 to 400 carbon atoms.

A particularly useful class of dispersants are the alkenylsuccinic derivatives, typically produced by the reaction of a long chain substituted alkenyl succinic compound, usually a substituted succinic anhydride, with a polyhydroxy or polyamino compound. The long chain group constituting the oleophilic portion of the molecule which confers solubility in the oil, is normally a polyisobutylene group. Many examples of this type of dispersant are well known commercially and in the literature. Exemplary patents describing such dispersants are U.S. Pat. Nos. 3,172,892; 3,219,666; 3,316,177 and 4,234,435. Other types of dispersants are described in U.S. Pat. Nos. 3,036,003; and 5,705,458.

Hydrocarbyl-substituted succinic acid compounds are popular dispersants. In particular, succinimide, succinate esters, or succinate ester amides prepared by the reaction of a hydrocarbon-substituted succinic acid compound preferably having at least 50 carbon atoms in the hydrocarbon substituent, with at least one equivalent of an alkylene amine are particularly useful.

Succinimides are formed by the condensation reaction between alkenyl succinic anhydrides and amines. Molar ratios can vary depending on the amine or polyamine. For example, the molar ratio of alkenyl succinic anhydride to TEPA can vary from 1:1 to 5:1.

Succinate esters are formed by the condensation reaction between alkenyl succinic anhydrides and alcohols or polyols. Molar ratios can vary depending on the alcohol or polyol used. For example, the condensation product of an alkenyl succinic anhydride and pentaerythritol is a useful dispersant.

Succinate ester amides are formed by condensation reaction between alkenyl succinic anhydrides and alkanol amines. For example, suitable alkanol amines include ethoxylated polyalkylpolyamines, propoxylated polyalkylpolyamines and polyalkenylpolyamines such as polyethylene polyamines. One example is propoxylated hexamethylenediamine.

The molecular weight of the alkenyl succinic anhydrides will typically range between 800 and 2,500. The above products can be post-reacted with various reagents such as sulfur, oxygen, formaldehyde, carboxylic acids such as oleic acid, and boron compounds such as borate esters or highly borated dispersants. The dispersants can be borated with from 0.1 to 5 moles of boron per mole of dispersant reaction product.

Mannich base dispersants are made from the reaction of alkylphenols, formaldehyde, and amines. Process aids and catalysts, such as oleic acid and sulfonic acids, can also be part of the reaction mixture. Molecular weights of the alkylphenols range from 800 to 2,500.

Typical high molecular weight aliphatic acid modified Mannich condensation products can be prepared from high molecular weight alkyl-substituted hydroxyaromatics or HN(R)₂ group-containing reactants.

Examples of high molecular weight alkyl-substituted hydroxyaromatic compounds are polypropylphenol, polybutylphenol, and other polyalkylphenols. These polyalkylphenols can be obtained by the alkylation, in the presence of an alkylating catalyst, such as BF₃, of phenol with high molecular weight polypropylene, polybutylene, and other polyalkylene compounds to give alkyl substituents on the benzene ring of phenol having an average 600-100,000 molecular weight.

Examples of HN(R)₂ group-containing reactants are alkylene polyamines, principally polyethylene polyamines. Other representative organic compounds containing at least one HN(R)₂ group suitable for use in the preparation of Mannich condensation products are well known and include the mono- and di-amino alkanes and their substituted analogs, e.g., ethylamine and diethanol amine; aromatic diamines, e.g., phenylene diamine, diamino naphthalenes; heterocyclic amines, e.g., morpholine, pyrrole, pyrrolidine, imidazole, imidazolidine, and piperidine; melamine and their substituted analogs.

Examples of alkylene polyamine reactants include ethylenediamine, diethylene triamine, triethylene tetraamine, tetraethylene pentaamine, pentaethylene hexamine, hexaethylene heptaamine, heptaethylene octaamine, octaethylene nonaamine, nonaethylene decamine, and decaethylene undecamine and mixture of such amines having nitrogen contents corresponding to the alkylene polyamines, in the formula H₂N—(Z—NH—)H, mentioned before, Z is a divalent ethylene and n is 1 to 10 of the foregoing formula. Corresponding propylene polyamines such as propylene diamine and di-, tr-, tetra-, pentapropylene tri-, tetra-, penta- and hexaamines are also suitable reactants. The alkylene polyamines are usually obtained by the reaction of ammonia and dihalo alkanes, such as dichloro alkanes. Thus the alkylene polyamines obtained from the reaction of 2 to 11 moles of ammonia with 1 to 10 moles of dichloroalkanes having 2 to 6 carbon atoms and the chlorines on different carbons are suitable alkylene polyamine reactants.

Aldehyde reactants useful in the preparation of the high molecular products useful in this disclosure include the aliphatic aldehydes such as formaldehyde (also as paraformaldehyde and formalin), acetaldehyde and aldol (β-hydroxybutyraldehyde). Formaldehyde or a formaldehyde-yielding reactant is preferred.

Preferred dispersants include borated and non-borated succinimides, including those derivatives from mono-succinimides, bis-succinimides, and/or mixtures of mono- and bis-succinimides, wherein the hydrocarbyl succinimide is derived from a hydrocarbylene group such as polyisobutylene having a Mn of from 500 to 5000 or a mixture of such hydrocarbylene groups. Other preferred dispersants include succinic acid-esters and amides, alkylphenol-polyamine-coupled Mannich adducts, their capped derivatives, and other related components. Such additives may be used in an amount of 0.1 to 20 wt %, preferably 0.1 to 8 wt %, more preferably 1 to 6 wt % (on an as-received basis) based on the weight of the total lubricant.

### Pour Point Depressants

Conventional pour point depressants (also known as lube oil flow improvers) may also be present. Pour point depressant may be added to lower the minimum temperature at which the fluid will flow or can be poured. Examples of suitable pour point depressants include alkylated naphthalenes polymethacrylates, polyacrylates, polyarylamides, condensation products of haloparaffin waxes and aromatic compounds, vinyl carboxylate polymers, and terpolymers of dialkylfumarates, vinyl esters of fatty acids and allyl vinyl ethers. Such additives may be used in amount of 0.0 to 0.5 wt %, preferably 0 to 0.3 wt %, more preferably 0.001 to 0.1 wt % on an as-received basis.

### Corrosion Inhibitors/Metal Deactivators

Corrosion inhibitors are used to reduce the degradation of metallic parts that are in contact with the lubricating oil composition. Suitable corrosion inhibitors include aryl thiazines, alkyl substituted dimercapto thiodiazoles thiadiazoles and mixtures thereof. Such additives may be used in an amount of 0.01 to 5 wt %, preferably 0.01 to 1.5 wt %, more preferably 0.01 to 0.2 wt %, still more preferably 0.01 to 0.1 wt % (on an as-received basis) based on the total weight of the lubricating oil composition.

### Seal Compatibility Additives

Seal compatibility agents help to swell elastomeric seals by causing a chemical reaction in the fluid or physical change in the elastomer. Suitable seal compatibility agents for lubricating oils include organic phosphates, aromatic esters, aromatic hydrocarbons, esters (butylbenzyl phthalate, for example), and polybutenyl succinic anhydride and sulfolane-type seal swell agents such as Lubrizol 730-type seal swell additives. Such additives may be used in an amount of 0.01 to 3 wt %, preferably 0.01 to 2 wt % on an as-received basis.

### Anti-Foam Agents

Anti-foam agents may advantageously be added to lubricant compositions. These agents retard the formation of stable foams. Silicones and organic polymers are typical anti-foam agents. For example, polysiloxanes, such as silicon oil or polydimethyl siloxane, provide antifoam properties. Anti-foam agents are commercially available and may be used in conventional minor amounts along with other additives such as demulsifiers; usually the amount of these additives combined is less than 1 percent, preferably 0.001 to 0.5 wt %, more preferably 0.001 to 0.2 wt %, still more preferably 0.0001 to 0.15 wt % (on an as-received basis) based on the total weight of the lubricating oil composition.

### Inhibitors and Antirust Additives

Anti-rust additives (or corrosion inhibitors) are additives that protect lubricated metal surfaces against chemical attack by water or other contaminants. One type of anti-rust additive is a polar compound that wets the metal surface preferentially, protecting it with a film of oil. Another type of anti-rust additive absorbs water by incorporating it in a water-in-oil emulsion so that only the oil touches the surface. Yet another type of anti-rust additive chemically adheres to the metal to produce a non-reactive surface. Examples of suitable additives include zinc dithiophosphates, metal phenolates, basic metal sulfonates, fatty acids and amines. Such additives may be used in an amount of 0.01 to 5 wt %, preferably 0.01 to 1.5 wt % on an as-received basis.

In addition to the ZDDP anti-wear additives which are essential components of the present disclosure, other anti-wear additives can be present, including zinc dithiocarbamates, molybdenum dialkyldithiophosphates, molybdenum dithiocarbamates, other organo molybdenum-nitrogen complexes, sulfurized olefins, etc.

The term “organo molybdenum-nitrogen complexes” embraces the organo molybdenum-nitrogen complexes described in U.S. Pat. No. 4,889,647. The complexes are reaction products of a fatty oil, dithanolamine and a molybdenum source. Specific chemical structures have not been assigned to the complexes. U.S. Pat. No. 4,889,647 reports an infrared spectrum for a typical reaction product of that disclosure; the spectrum identifies an ester carbonyl band at 1740 cm⁻¹ and an amide carbonyl band at 1620 cm⁻¹. The fatty oils are glyceryl esters of higher fatty acids containing at least 12 carbon atoms up to 22 carbon atoms or more. The molybdenum source is an oxygen-containing compound such as ammonium molybdates, molybdenum oxides and mixtures.

Other organo molybdenum complexes which can be used in the present disclosure are tri-nuclear molybdenum-sulfur compounds described in EP 1 040 115 and WO 99/31113 and the molybdenum complexes described in U.S. Pat. No. 4,978,464.

In the above detailed description, the specific embodiments of this disclosure have been described in connection with its preferred embodiments. However, to the extent that the above description is specific to a particular embodiment or a particular use of this disclosure, this is intended to be illustrative only and merely provides a concise description of the exemplary embodiments. Accordingly, the disclosure is not limited to the specific embodiments described above, but rather, the disclosure includes all alternatives, modifications, and equivalents falling within the true scope of the appended claims. Various modifications and variations of this disclosure will be obvious to a worker skilled in the art and it is to be understood that such modifications and variations are to be included within the purview of this application and the spirit and scope of the claims.

### EXAMPLES

All starting materials and solvents were purchased from commercial sources and used without further purification. All reactions were carried out in oven-dried glassware. ¹H and ¹³C NMR spectra were acquired in CDCl₃ on a Bruker 400 MHz spectrometer. ¹H and ¹³C chemical shifts (5) are given in ppm relative to the residual protonated chloroform peak. Fourier transform infrared (FTIR) spectra were recorded on a Nicolet Nexus 470 spectrometer.

### Example 1

### Synthesis of tetraoctylammonium bis(trifluoromethanesulfonyl)imide

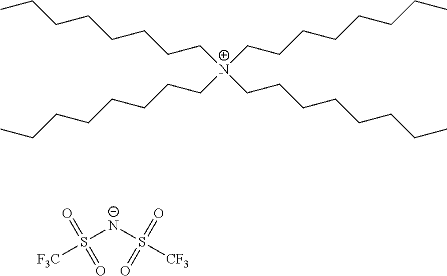 [SMILES: CCCCCCCC[N+](CCCCCCCC)(CCCCCCCC)CCCCCCCC ; O=S(=O)([N-]S(=O)(=O)C(F)(F)F)C(F)(F)F]

A solution of lithium bis(trifluoromethanesulfonyl)imide (2.30 grams, 8.03 mmol) in 3.3 milliliters of de-ionized water and 6.6 milliliters of acetone was added dropwise to a solution of tetraoctylammonium bromide (4.00 grams, 7.30 mmol) in 15 milliliters of acetone and 5 milliliters of de-ionized water. The reaction mixture was stirred at room temperature for 12 hours. After the reaction was completed, acetone was removed by rotary evaporation. Dichloromethane (30 milliliters) was added and the organic layer was washed with de-ionized water (3×20 milliliters). The organic layer was then dried over MgSO₄, filtered, and concentrated by rotary evaporation to afford the product as a clear liquid (5.20 grams, 96% yield). ¹H NMR (400 MHz, CDCl₃) δ 3.15 (m, 8H); 1.61 (m, 8H); 1.42-1.15 (m, 40H); 0.89 (t, 12H). ¹³C NMR (100 MHz, CDCl₃) δ 121.5, 118.3, 58.58, 31.54, 28.88, 28.86, 26.03, 22.52, 21.74, 13.96. IR (film) 2956, 2928, 2858, 1484, 1468, 1352, 1225, 1194, 1137, 1057 cm⁻¹.

### Example 2

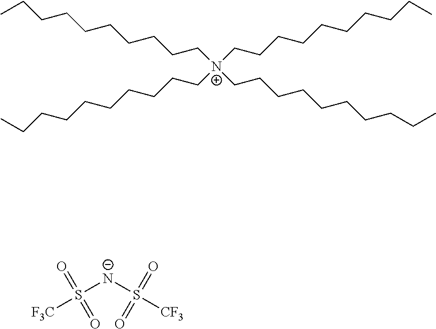 [SMILES: CCCCCCCCCC[N+](CCCCCCCCCC)(CCCCCCCCCC)CCCCCCCCCC ; O=S(=O)([N-]S(=O)(=O)C(F)(F)F)C(F)(F)F]

### Synthesis of tetradecylammonium bis(trifluoromethanesulfonyl)imide

A solution of lithium bis(trifluoromethanesulfonyl)imide (1.92 grams, 6.67 mmol) in 10 milliliters of acetone was added dropwise to a solution of tetradecylammonium bromide (4.00 grams, 6.07 mmol) in 80 milliliters of acetone at 50° C. until all components were completely dissolved. The reaction mixture was stirred at room temperature for 16 hours. After the reaction was completed, acetone was removed by rotary evaporation. Dichloromethane (30 milliliters) was added and the organic layer was washed with de-ionized water (3×20 milliliters). The organic layer was then dried over MgSO₄, filtered, and concentrated by rotary evaporation to afford the product as a clear liquid (4.79 grams, 92% yield). ¹H NMR (400 MHz, CDCl₃) δ 3.14 (m, 8H); 1.61 (m, 8H); 1.44-1.22 (m, 56H); 0.88 (t, 12H). ¹³C NMR (100 MHz, CDCl₃) δ 121.5, 118.3, 58.73, 31.81, 29.34, 29.25, 29.20, 28.94, 26.09, 22.61, 21.81, 14.02. IR (film) 2926, 2856, 1468, 1352, 1225, 1196, 1137, 1058 cm⁻¹.

### Example 3

### Lube Properties and Thermal Stability of Ionic Liquids

The kinematic viscosity (Kv) of the ionic liquid products of Examples 1 and 2 was measured using ASTM standards D-445 and reported at temperatures of 100° C. (Kv at 100° C.) or 40° C. (Kv at 40° C.). The viscosity index (VI) was measured according to ASTM standard D-2270 using the measured kinematic viscosities for each ionic liquid product.

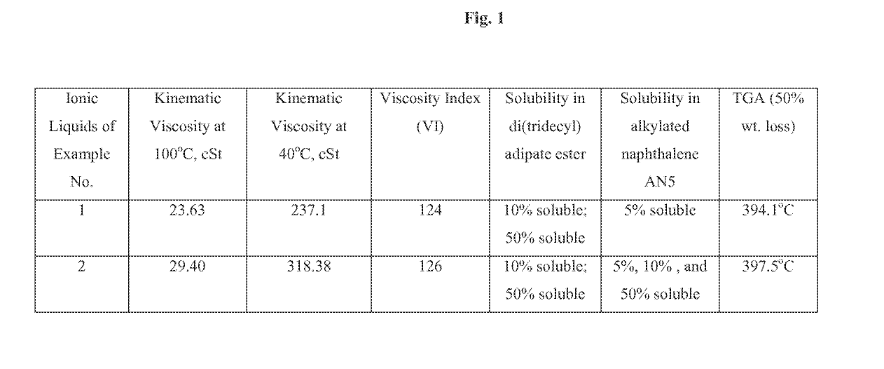

The thermal stability of the ionic liquids of Examples 1 and 2 was evaluated using TGA. The 50% wt. loss is shown in FIG. 1. All ionic liquids showed 50% wt. loss at greater than 390° C. Thus, these results show that the ionic liquids of this disclosure are thermally stable. Properties of the ionic liquids (Kv at 100° C., Kv at 40° C., viscosity index, solubility in di(tridecyl) adipate ester, solubility in alkylated naphthalene AN5, and TGA) are set forth in FIG. 1.

### Example 4

### Synthesis of 1-methyl-3-decylimidazolium bromide

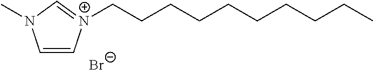 [SMILES: CCCCCCCCCC[n+]1ccn(C)c1 ; [Br-]]

To a solution of 1-methylimidazole (10.00 g, 121.8 mmol) in toluene (50 milliliters) was added 1-bromodecane (29.63 grams, 134.0 mmol) drop wise at room temperature. The reaction mixture was refluxed for 12 hours. A white precipitate formed after cooling the reaction mixture to room temperature. After filtration, the product was washed with hexanes (4×50 milliliters). The isolated product was then further dried in vacuum at 50° C. for 2 hours to completely remove any residual solvents or moisture. The product was obtained as a slightly yellow viscous liquid (35.1 grams, 95% yield). ¹H NMR (400 MHz, CDCl₃) δ 10.38 (s, 1H); 7.51 (t, 1H); 7.35 (t, 1H); 4.28 (t, 2H); 4.09 (s, 3H); 1.87 (m, 2H); 1.31-1.19 (m, 14H); 0.82 (t, 3H). IR (film) 3139, 3064, 2955, 2924, 2854, 1571, 1466, 1378, 1170 cm⁻¹.

### Example 5

### Synthesis of 1-methyl-3-decylimidazolium bis(trifluoromethanesulfonyl)imide

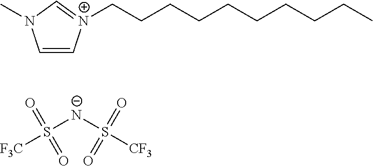 [SMILES: CCCCCCCCCC[n+]1ccn(C)c1 ; O=S(=O)([N-]S(=O)(=O)C(F)(F)F)C(F)(F)F]

A solution of lithium bis(trifluoromethanesulfonyl)imide (3.26 grams, 11.37 mmol) in 10 milliliters of de-ionized water was added drop wise to a solution of 1-methyl-3-decylimidazolium bromide (3.00 grams, 9.89 mmol) in 10 milliliters of de-ionized water. The reaction mixture was stirred at room temperature for 16 hours. After the reaction was completed, dichloromethane (30 milliliters) was added and the organic layer was washed with de-ionized water (3×10 milliliters). The organic layer was then dried over MgSO₄, filtered, and concentrated by rotary evaporation to afford the ionic liquid as slightly yellow liquid (4.78 grams, 96% yield). ¹H NMR (400 MHz, CDCl₃) δ 8.75 (s, 1H); 7.31 (t, 1H); 7.30 (t, 1H); 4.16 (t, 2H) 3.94 (s, 3H); 1.84 (m, 2H); 1.31-1.25 (m, 14H); 0.88 (t, 3H). IR (film) 3156, 3121, 2928, 2857, 1573, 1468, 1352, 1194, 1137, 1059 cm⁻¹.

### Example 6

### Synthesis of 1-methyl-3-hexadecylimidazolium bromide

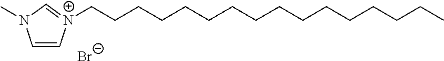 [SMILES: CCCCCCCCCCCCCCCC[n+]1ccn(C)c1 ; [Br-]]

To a solution of 1-methylimidazole (5.00 grams, 60.9 mmol) in toluene (50 milliliters) was added 1-bromohexadecane (20.45 g, 66.99 mmol) dropwise at room temperature. The reaction mixture was refluxed for 12 hours. A white precipitate formed after cooling the reaction mixture to room temperature. After filtration, the product was washed with hexanes (4×50 milliliters). The isolated product was then further dried in vacuo at 50° C. for 24 hours to completely remove any residual solvents or moisture. The product was obtained as a white solid (22.8 grams, 97% yield). ¹H NMR (400 MHz, CDCl₃) δ 10.74 (s, 1H); 7.27 (t, 1H); 7.21 (t, 1H); 4.32 (t, 2H); 4.14 (s, 3H); 1.92 (m, 2H); 1.36-1.25 (m, 26H); 0.88 (t, 3H). IR (film) 3140, 3054, 2925, 2854, 1571, 1467, 1265, 1170 cm⁻¹.

### Example 7

### Synthesis of 1-methyl-3-hexadecylimidazolium bis(trifluoromethanesulfonyl)imide

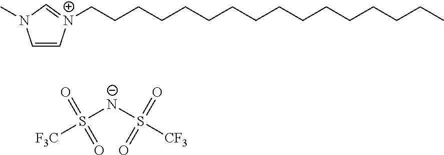 [SMILES: CCCCCCCCCCCCCCCC[n+]1ccn(C)c1 ; O=S(=O)([N-]S(=O)(=O)C(F)(F)F)C(F)(F)F]

A solution of lithium bis(trifluoromethanesulfonyl)imide (2.50 grams, 8.71 mmol) in de-ionized water (7.5 milliliters) was added dropwise to a solution of 1-methyl-3-hexadecylimidazolium bromide (3.00 grams, 7.92 mmol) in de-ionized water (7.5 milliliters) and acetone (5 milliliters). The reaction mixture was stirred at room temperature for 16 hours. After the reaction was completed, acetone was removed by rotary evaporation. Dichloromethane (30 milliliters) was added to the reaction mixture and the organic layer was washed with de-ionized water (3×10 milliliters). The organic layer was then dried over MgSO₄, filtered, and concentrated by rotary evaporation to afford the product as a white solid (4.42 grams, 95% yield). ¹H NMR (400 MHz, CDCl₃) δ 8.90 (s, 1H); 7.24 (t, 1H); 7.23 (t, 1H); 4.19 (t, 2H); 3.98 (s, 3H); 1.87 (m, 2H); 1.33-1.25 (m, 26H); 0.87 (t, 3H). IR (film) 3154, 3098, 2926, 2855, 1572, 1467, 1351, 1266, 1226, 1198, 1136, 1059 cm⁻¹.

### Example 8

### Synthesis of 1-butyl-3-decylimidazolium bromide

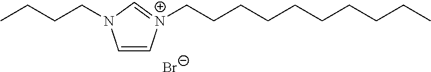 [SMILES: CCCCCCCCCC[n+]1ccn(CCCC)c1 ; [Br-]]

To a solution of 1-butylimidazole (10.00 grams, 80.52 mmol) in toluene (50 milliliters) was added 1-bromodecane (23.15 grams, 104.67 mmol) dropwise at room temperature. The reaction mixture was refluxed 16 hours. After cooling to room temperature, toluene was decanted and the crude product was washed with hexanes (3×100 milliliters). The isolated product was then further purified by vacuum distillation to completely remove any residual solvents, moisture, and starting materials. The product was obtained as white solid (25.0 grams, 90% yield). ¹H NMR (400 MHz, CDCl₃) δ 10.77 (s, 1H); 7.33 (t, 1H); 7.30 (t, 1H); 4.38 (m, 4H); 1.92 (m, 4H); 1.45-1.22 (m, 16H); 0.98 (t, 3H); 0.88 (t, 3H). IR (film) 3130, 3061, 2957, 2926, 2855, 1563, 1466, 1378, 1165 cm⁻¹

### Example 9

### Synthesis of 1-butyl-3-decylimidazolium bis(trifluoromethanesulfonyl)imide

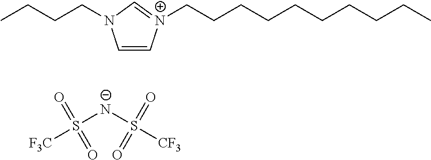 [SMILES: CCCCCCCCCC[n+]1ccn(CCCC)c1 ; O=S(=O)([N-]S(=O)(=O)C(F)(F)F)C(F)(F)F]

A solution of lithium bis(trifluoromethanesulfonyl)imide (3.02 grams, 10.50 mmol) in 10 milliliters of de-ionized water was added dropwise to a solution of 1-butyl-3-decylimidazolium bromide (3.00 grams, 9.55 mmol) in 30 milliliters of de-ionized water. The reaction mixture was stirred at room temperature for 12 hours. After the reaction was completed, dichloromethane (30 milliliters) was added to the reaction mixture and the organic layer was washed with de-ionized water (3×20 milliliters). The organic layer was then dried over MgSO₄, filtered, and concentrated by rotary evaporation to afford the product as slightly yellow liquid (4.60 grams, 97% yield). ¹H NMR (400 MHz, CDCl₃) δ 8.86 (s, 1H); 7.30 (t, 1H); 7.28 (t, 1H); 4.20 (m, 4H); 1.86 (m, 4H); 1.40-1.20 (m, 16H); 0.97 (t, 3H); 0.88 (t, 3H). IR (film) 3148, 2929, 2858, 1565, 1468, 1352, 1226, 1195, 1136, 1058 cm⁻¹.

### Example 10

### Synthesis of 1-butyl-3-hexadecylimidazolium bromide

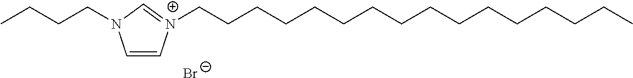 [SMILES: CCCCCCCCCCCCCCCC[n+]1ccn(CCCC)c1 ; [Br-]]

To a solution of 1-butylimidazole (5.00 grams, 40.26 mmol) in toluene (50 milliliters) was added 1-bromohexadecane (13.52 g, 44.28 mmol) dropwise at room temperature. The reaction mixture was refluxed for 16 hours. After cooling to room temperature, toluene was decanted and the crude product washed with hexanes (3×100 milliliters). The isolated product was then further purified by vacuum distillation to completely remove any residual solvents, moisture, and starting materials. The product was obtained as a white solid (15.9 grams, 92% yield). ¹H NMR (400 MHz, CDCl₃) δ 10.70 (s, 1H); 7.36 (t, 1H); 7.32 (t, 1H); 4.38 (m, 4H); 1.92 (m, 4H); 1.46-1.20 (m, 28H); 0.99 (t, 3H); 0.88 (t, 3H). IR (film) 3150, 3035, 2957, 2925, 2854, 1562, 1466, 1378, 1264, 1165 cm⁻¹.

### Example 11

### Synthesis of 1-butyl-3-hexadecylimidazolium bis(trifluoromethanesulfonyl)imide

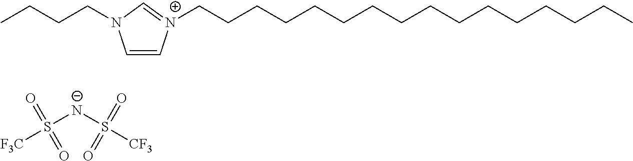 [SMILES: CCCCCCCCCCCCCCCC[n+]1ccn(CCCC)c1 ; O=S(=O)([N-]S(=O)(=O)C(F)(F)F)C(F)(F)F]

A solution of lithium bis(trifluoromethanesulfonyl)imide (3.02 grams, 10.50 mmol) in 10 milliliters of de-ionized water was added dropwise to a solution of 1-butyl-3-hexadecylimidazolium bromide (3.0 grams, 6.98 mmol) in de-ionized water (20 milliliters) and acetone (1 milliliters). The reaction mixture was stirred at room temperature for 12 hours. After the reaction was completed, dichloromethane (30 milliliters) was added to the reaction mixture and the organic layer was washed with de-ionized water (3×20 milliliters). The organic layer was then dried over MgSO₄, filtered, and concentrated by rotary evaporation to afford the product as a clear liquid (3.95 grams, 90% yield). ¹H NMR (400 MHz, CDCl₃) δ 8.91 (s, 1H); 7.22 (t, 1H); 7.21 (t, 1H); 4.20 (m, 4H); 1.90 (m, 4H); 1.40-1.19 (m, 28H); 0.98 (t, 3H); 0.88 (t, 3H). IR (film) 3148, 3114, 2925, 2854, 1564, 1467, 1352, 1226, 1196, 1136, 1059 cm¹.

### Example 12

### Synthesis of 1-benzyl-3-decylimidazolium bromide

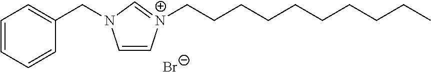 [SMILES: CCCCCCCCCC[n+]1ccn(Cc2ccccc2)c1 ; [Br-]]

To a solution of 1-benzylimidazole (10.00 grams, 63.21 mmol) in toluene (150 milliliters) was added 1-bromodecane (18.17 grams, 82.17 mmol) dropwise at room temperature. The reaction mixture was refluxed for 16 hours. After cooling to room temperature, toluene was decanted and the crude product washed with toluene (3×100 milliliters). The isolated product was then further purified by vacuum distillation to completely remove any residual solvents, moisture, and starting materials. The product was obtained as a slightly yellow liquid (21.6 grams, 90% yield). ¹H NMR (400 MHz, CDCl₃) δ 10.68 (s, 1H); 7.52 (m, 2H); 7.38 (m, 5H); 5.64 (s, 2H); 4.29 (t, 2H); 1.91 (m, 2H); 1.38-1.18 (m, 14H); 0.88 (t, 3H). IR (film) 3127, 3063, 2955, 2925, 2854, 1606, 1560, 1497, 1457, 1377, 1208, 1157, 1079, 1030 cm⁻¹.

### Example 13

### Synthesis of 1-benzyl-3-decylimidazolium bis(trifluoromethanesulfonyl)imide

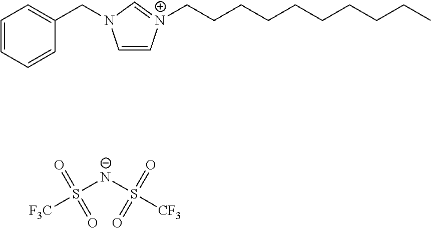 [SMILES: CCCCCCCCCC[n+]1ccn(Cc2ccccc2)c1 ; O=S(=O)([N-]S(=O)(=O)C(F)(F)F)C(F)(F)F]

A solution of lithium bis(trifluoromethanesulfonyl)imide (3.02 grams, 10.50 mmol) in acetone (9 milliliters) and de-ionized water (3 milliliters) was added dropwise to a solution of 1-benzyl-3-decylimidazolium bromide (3.46 grams, 9.12 mmol) in acetone (15 milliliters) and de-ionized water (5 milliliters). The reaction mixture was stirred at room temperature for 4 hours. Acetone was removed upon reaction completion. Dichloromethane (30 milliliters) was added to the reaction mixture, and the organic layer was washed with de-ionized water (3×20 milliliters). The organic layer was then dried over MgSO₄, filtered, and concentrated by rotary evaporation to afford the product as a clear liquid (5.02 grams, 95% yield). ¹H NMR (400 MHz, CDCl₃) δ 8.95 (s, 1H); 7.42 (m, 3H); 7.37 (m, 2H); 7.22 (t, 1H); 7.18 (t, 1H); 5.35 (s, 2H); 4.18 (t, 2H); 1.87 (m, 2H); 1.33-1.20 (m, 14H); 0.87 (t, 3H). ¹³C NMR (100 MHz, CDCl₃) δ 135.24, 132.47, 129.69, 129.55, 128.82, 122.54, 122.30, 121.44, 118.24, 53.54, 50.29, 31.80, 30.03, 29.35, 29.25, 29.18, 28.80, 26.07, 22.62, 14.05. IR (film) 3147, 2928, 2857, 1562, 1499, 1459, 1352, 1197, 1136, 1058 cm⁻¹.

### Example 14

### Lube Properties and Thermal Stability of Ionic Liquids

The kinematic viscosity (Kv) of the ionic liquid products of Examples 5, 7, 9, 11 and 13 was measured using ASTM standards D-445 and reported at temperatures of 100° C. (Kv at 100° C.) or 40° C. (Kv at 40° C.). The viscosity index (VI) was measured according to ASTM standard D-2270 using the measured kinematic viscosities for each product.

The ionic liquids of Examples 5, 7, 9, 11 and 13 have good viscosity index. All five ionic liquids are highly soluble in di(tridecyl) adipate ester base stocks.

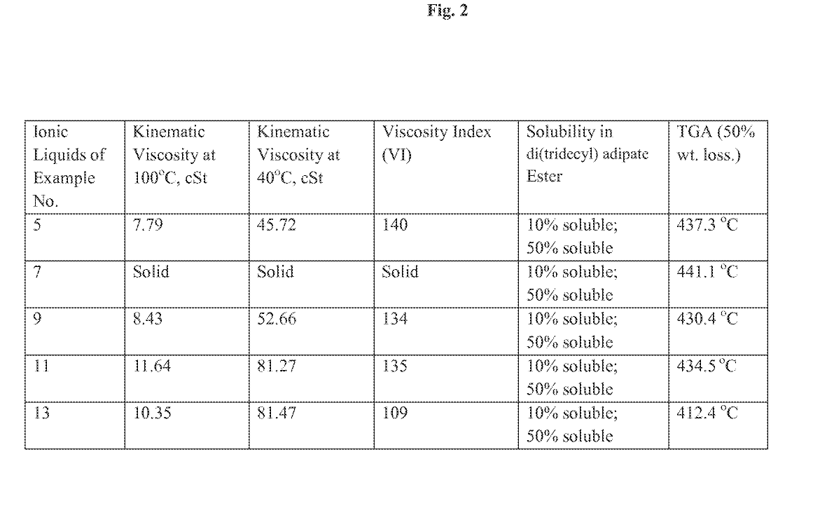

The thermal stability of the ionic liquids of Examples 5, 7, 9, 11 and 13 was evaluated using TGA. The 50% wt. loss is shown in FIG. 2. All ionic liquids showed 50% wt. loss at greater than 400° C., while PAO 4 showed 50% wt. loss at 261° C. under same conditions. Thus, the results show that the ionic liquids of this disclosure are substantially more stable than hydrocarbon fluid PAO 4. Properties of the ionic liquids (Kv at 100° C., Kv at 40° C., viscosity index, solubility in di(tridecyl) adipate ester, solubility in alkylated naphthalene AN5, and TGA) are set forth in FIG. 2.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

What is claimed is:

1. A composition comprising:
(i) an ionic liquid alkylammonium salt represented by the formula
R₄N⁺,[F₃CS(O)₂]₂N⁻ (1)
wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein said ionic liquid alkylammonium salt has a structure sufficient to exhibit at least partial solubility in one or more Group I-V base stocks; or
(ii) an ionic liquid imidazolium salt represented by the formula 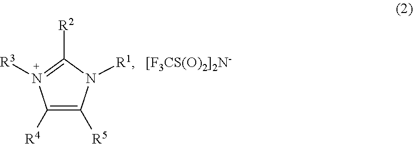
wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.

2. The composition of claim 1 wherein the ionic liquid alkylammonium salt of formula (1) is represented by the formula
[C₈H₁₇]₄N⁺,[F₃CS(O)₂]₂N⁻ or
[C₁₀H₂₁]₄N⁺,[F₃CS(O)₂]₂N⁻,
and wherein the ionic liquid imidazolium salt of formula (2) is represented by the formula

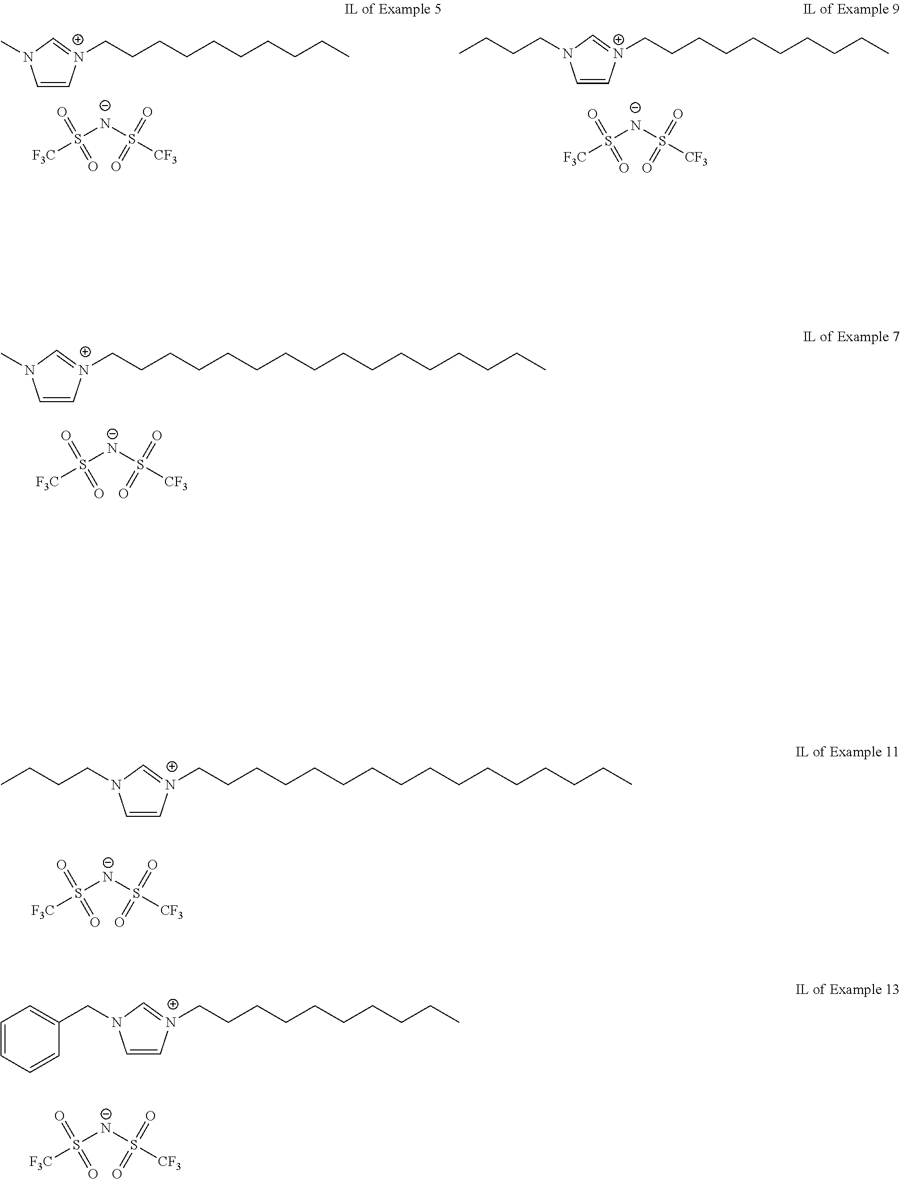

3. The composition of claim 1 wherein, in formula (1), R is independently C₃H₇, C₄H₉, C₅H₁₁, C₆H₁₃, C₈H₁₇, C₁₀H₂₁, C₁₂H₂₅, C₁₄H₂₉ or C₁₆H₃₃; and wherein, in formula (2), R¹ is CH₃, C₂H₅, C₃H₇, C₄H₉, C₅H₁₁, C₆H₁₃, C₈H₁₇, C₁₀H₂₁, or C₁₂H₂₅, and R³ is C₁₀H₂₁, C₁₂H₂₅, C₁₄H₂₉, C₁₆H₃₃, C₁₈H₃₇, or C₂₀H₄₁.

4. The composition of claim 1 wherein said ionic liquid alkylammonium salt of formula (1) has a solubility in one or more Group I-V base stocks of at least 5%, and said ionic liquid imidazolium salt of formula (2) has a solubility in one or more Group I-V base stocks of at least 5%.

5. The composition of claim 1 wherein said one or more Group I-V base stocks comprises a Group V base stock.

6. The composition of claim 1 which has an onset of thermal decomposition temperature greater than 250° C.

7. The composition of claim 1 having a viscosity (Kv₁₀₀) from 2 to 400 at 100° C., and a viscosity index (VI) from 100 to 300.

8. A lubricating oil base stock comprising:
(i) an ionic liquid alkylammonium salt represented by the formula
R₄N⁺,[F₃CS(O)₂]₂N⁻ (1)
wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein said ionic liquid alkylammonium salt has a structure sufficient to exhibit at least partial solubility in one or more Group I-V base stocks; or
(ii) an ionic liquid imidazolium salt represented by the formula 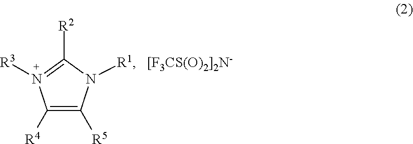
wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.

9. A lubricating oil comprising a lubricating oil base stock as a major component, and an ionic liquid alkylammonium salt cobase stock or an ionic liquid imidazolium salt cobase stock, as a minor component; wherein said ionic liquid alkylammonium salt is represented by the formula
R₄N⁺,[F₃CS(O)₂]₂N⁻ (1)
wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein said ionic liquid alkylammonium salt has a structure sufficient to exhibit at least partial solubility in one or more Group I-V base stocks; and said ionic liquid imidazolium salt is represented by the formula 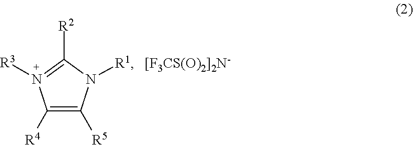
wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.

10. The lubricating oil of claim 9 wherein the lubricating oil base stock comprises a Group I, II, III, IV or V base oil stock.

11. The lubricating oil of claim 9 wherein the lubricating oil base stock is present in an amount from 50 weight percent to 99 weight percent, and the ionic liquid alkylammonium salt cobase stock or the ionic liquid imidazolium salt cobase stock is present in an amount from 1 weight percent to 50 weight percent, based on the total weight of the lubricating oil.

12. The lubricating oil of claim 9 wherein the ionic liquid alkylammonium salt of formula (1) is represented by the formula
[C₈H₁₇]₄N⁺,[F₃CS(O)₂]₂N⁻ or
[C₁₀H₂₁]₄N⁺,[F₃CS(O)₂]₂N⁻,
and wherein the ionic liquid imidazolium salt of formula (2) is represented by the formula

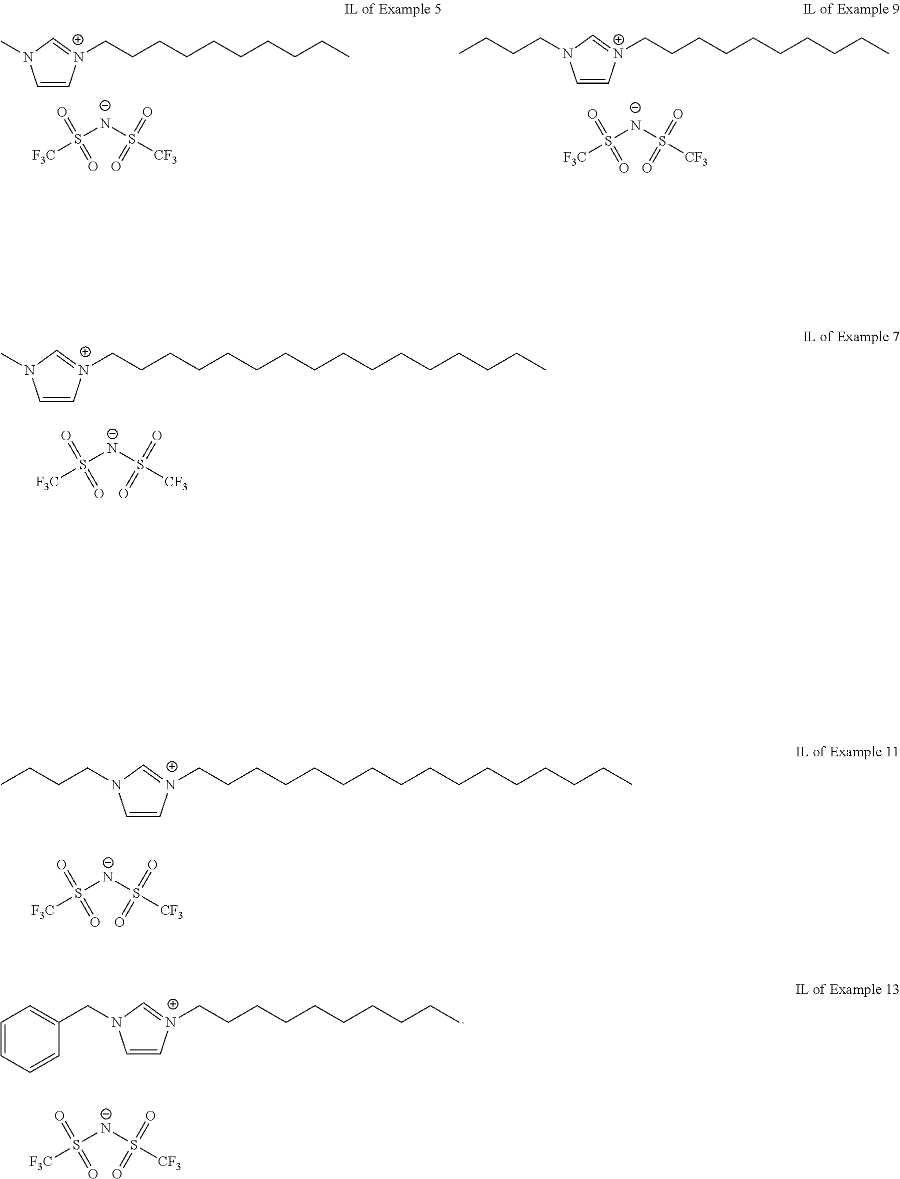

13. The lubricating oil of claim 9 wherein, in formula (1), R is independently C₃H₇, C₄H₉, C₅H₁₁, C₆H₁₃, C₈H₁₇, C₁₀H₂₁, C₁₂H₂₅, C₁₄H₂₉ or C₁₆H₃₃; and wherein, in formula (2), R¹ is CH₃, C₂H₅, C₃H₇, C₄H₉, C₅H₁₁, C₆H₁₃, C₈H₁₇, C₁₀H₂₁, or C₁₂H₂₅, and R³ is C₁₀H₂₁, C₁₂H₂₅, C₁₄H₂₉, C₁₆H₃₃, C₁₈H₃₇, or C₂₀H₄₁.

14. The lubricating oil of claim 9 wherein said ionic liquid alkylammonium salt of formula (1) has a solubility in one or more Group I-V base stocks of at least 5%, and said ionic liquid imidazolium salt of formula (2) has a solubility in one or more Group I-V base stocks of at least 5%.

15. The lubricating oil of claim 9 which further comprises one or more of a viscosity improver, antioxidant, detergent, dispersant, pour point depressant, corrosion inhibitor, metal deactivator, seal compatibility additive, anti-foam agent, inhibitor, and anti-rust additive.

16. A multifunctional functional fluid comprising:
(i) an ionic liquid alkylammonium salt represented by the formula
R₄N⁺,[F₃CS(O)₂]₂N⁻ (1)
wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein said ionic liquid alkylammonium salt has a structure sufficient to exhibit at least partial solubility in one or more Group I-V base stocks; or
(ii) an ionic liquid imidazolium salt represented by the formula 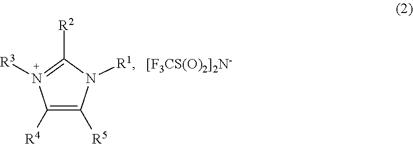
wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.

17. The multifunctional functional fluid of claim 16 wherein the ionic liquid alkylammonium salt of formula (1) is represented by the formula
[C₈H₁₇]₄N⁺,[F₃CS(O)₂]₂N⁻ or
[C₁₀H₂₁]₄N⁺,[F₃CS(O)₂]₂N⁻,
and wherein the ionic liquid imidazolium salt of formula (2) is represented by the formula

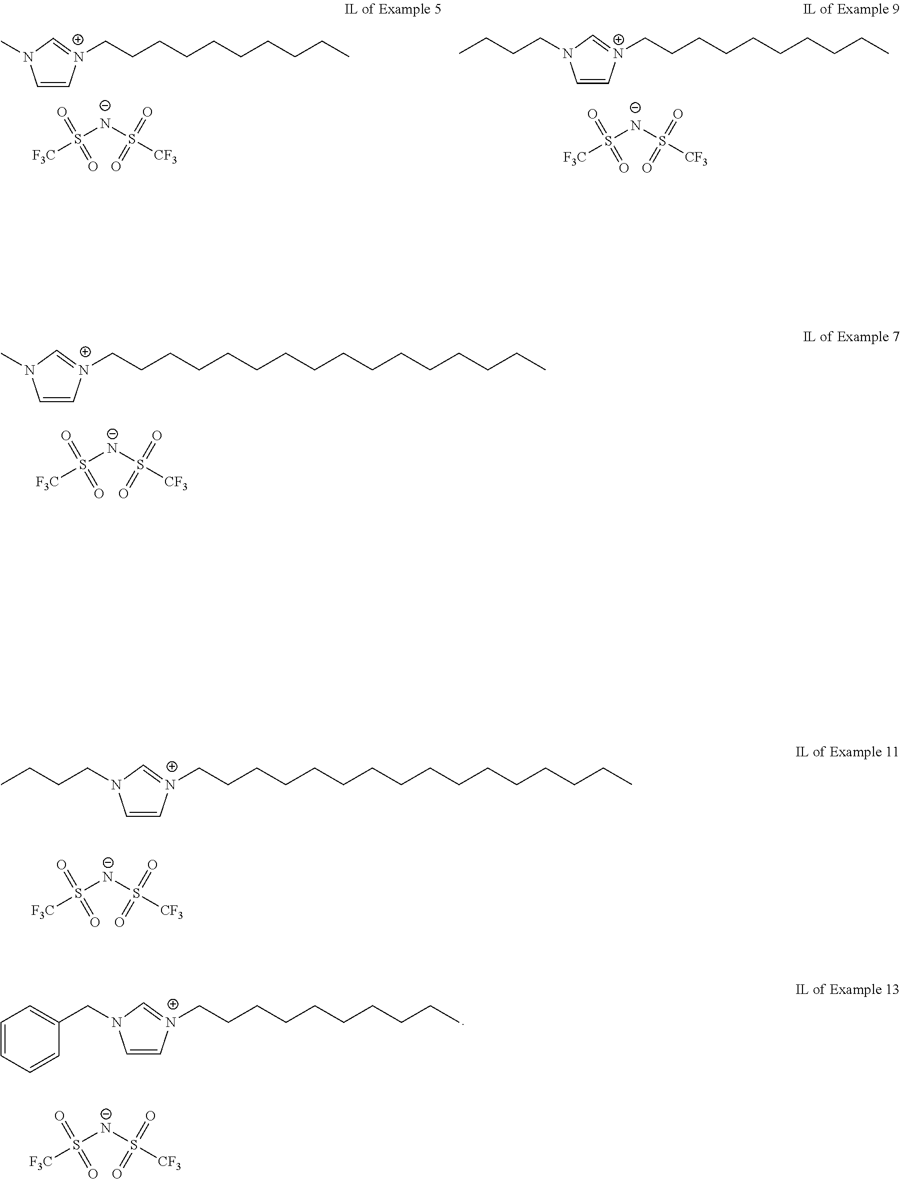

18. The multifunctional functional fluid of claim 16 wherein, in formula (1), R is independently C₃H₇, C₄H₉, C₅H₁₁, C₆H₁₃, C₈H₁₇, C₁₀H₂₁, C₁₂H₂₅, C₁₄H₂₉ or C₁₆H₃₃; and wherein, in formula (2), R¹ is CH₃, C₂H₅, C₃H₇, C₄H₉, C₅H₁₁, C₆H₁₃, C₈H₁₇, C₁₀H₂₁, or C₁₂H₂₅, and R³ is C₁₀H₂₁, C₁₂H₂₅, C₁₄H₂₉, C₁₆H₃₃, C₁₈H₃₇, or C₂₀H₄₁.

19. The multifunctional functional fluid of claim 16 wherein said ionic liquid alkylammonium salt of formula (1) has a solubility in one or more Group I-V base stocks of at least 5%, and said ionic liquid imidazolium salt of formula (2) has a solubility in one or more Group I-V base stocks of at least 5%.

20. A method for improving solubility of an ionic liquid in a lubricating oil by using as the lubricating oil a formulated oil comprising a lubricating oil base stock as a major component, and an ionic liquid alkylammonium salt cobase stock or an ionic liquid imidazolium salt cobase stock, as a minor component; wherein said ionic liquid alkylammonium salt is represented by the formula
R₄N⁺,[F₃CS(O)₂]₂N⁻ (1)
wherein R is independently C₁ to C₁₆ straight chain alkyl, branched chain alkyl, cycloalkyl, alkyl substituted cycloalkyl, cycloalkyl substituted alkyl, or, optionally, two R groups comprise a cyclic structure including the nitrogen atom and 4 to 12 carbon atoms; wherein said ionic liquid alkylammonium salt has a structure sufficient to exhibit at least partial solubility in one or more Group I-V base stocks; and said ionic liquid imidazolium salt is represented by the formula 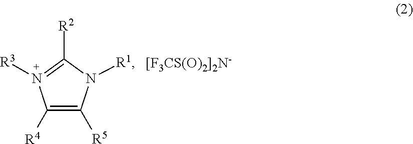
wherein R¹ and R³ are independently a C₁ to C₂₄ straight chain or branched chain alkyl group, a C₆ to C₁₀ aryl group, a C₇ to C₁₂ arylalkyl group, a C₇ to C₁₂ alkylaryl group, a C₂ to C₈ alkenyl group, a C₁ to C₈ alkoxy group, a C₂ to C₈ alkinyl group, or a C₂ to C₈ acyl group, provided at least one of R¹ and R³ is a C₁₀ to C₂₄ straight chain or branched chain alkyl group; R², R⁴ and R⁵ are hydrogen; wherein said ionic liquid imidazolium salt has a structure sufficient to exhibit at least partial solubility in one or more Group I-V base stocks.
